(19) United States
(12) Patent Application Publication
(10) Pub. No.: US 2021/0293794 A1
(43) Pub. Date: Sep. 23, 2021

(54) METABOLIC BIOMARKERS FOR THE IDENTIFICATION AND CHARACTERIZATION OF ALZHEIMERS DISEASE

(71) Applicant: UNIVERSITY OF HAWII CANCER CENTER, Honolulu, HI (US)

(72) Inventors: Wei Jia, Honolulu, HI (US); Matthias Arnold, Neuherberg, Bavaria (DE); Gabi Kastenmueller, Neuherberg, Bavaria (DE); Kwangsik Nho, Indianapolis, IN (US); Andrew J. Saykin, Indianapolis, IN (US); Siamak Mahmoudian Dehkordi, Raleigh, NC (US); Rima F. Kaddurah-Daouk, Belmont, MA (US); Gregory Louie, Durham, NC (US); Therese Koal, Innsbruck (AT)

(21) Appl. No.: 16/488,393

(22) PCT Filed: Feb. 24, 2018
(86) PCT No.: PCT/US2018/019602

§ 371 Date: Aug. 23, 2019

Related U.S. Application Data
(60) Provisional application No. 62/463,210, filed on Feb. 24, 2017.
(60) Provisional application No. 62/468,653, filed on Mar. 8, 2017.

(51) Int. Cl. G01N 33/53; G01N 33/68; C07J 1/00

(52) U.S. Cl. CPC G01N 33/5308; C07J 1/00; G01N 33/6896

(57) ABSTRACT

Embodiments of the present disclosure relate generally to the analysis of broad metabolic changes associated with neurological disorders. In particular, the present disclosure provides materials and methods relating to the use of metabolomics as a biochemical approach to identify peripheral metabolic changes and corresponding metabolic biomarkers of neurological disorders. Embodiments of the present disclosure include the use of bile acids and their derivatives as metabolic biomarkers to aid in the determination of whether a subject suffers from, or is at risk of developing, a neurological disorder, such as Alzheimer's Disease (AD).

### RELATED APPLICATIONS

This patent application is the U.S. National Stage Entry, under 35 U.S.C. § 371, of International Application Number PCT/US2018/019602, filed Feb. 24, 2018, which claims priority to and the benefit of U.S. Provisional Patent Application Ser. No. 62/463,210 filed Feb. 24, 2017, and U.S. Provisional Patent Application Ser. No. 62/468,653 filed Mar. 8, 2017, the entire contents of each of which are hereby incorporated by reference.

### GOVERNMENT FUNDING

The subject matter of this invention was made with Government support under Federal Grant Nos. R01AG046171, RF1AG051550, 3U01AG024904-09S4, P50NS053488, R01AG19771, P30AG10133, R01LM011360, P30AG10124, R00LM011384, R03AG054936, and K01AG049050. The government has certain rights in this invention.

### FIELD

Embodiments of the present disclosure relate generally to the analysis of broad metabolic changes associated with neurological disorders. In particular, the present disclosure provides materials and methods relating to the use of metabolomics as a biochemical approach to identify peripheral metabolic changes and corresponding metabolic biomarkers of neurological disorders. Embodiments of the present disclosure include the use of bile acids and their derivatives as metabolic biomarkers to aid in the determination of whether a subject suffers from, or is at risk of developing, a neurological disorder, such as Alzheimer's Disease (AD).

### BACKGROUND

Alzheimer's (AD) is a progressive neurodegenerative disorder, which currently has no cure or preventive therapy, and its symptomatic therapies are only modestly effective. The failure of hundreds of trials of disease-modifying therapeutics, including several targeting amyloid-beta (AP), highlights our incomplete knowledge of both cause of AD and mechanisms of cognitive failure. A large number of biochemical processes are affected in disease including glucose cholesterol mitochondrial energetics and lipid metabolism. Several metabolic changes happen early and are noted in blood prior to symptoms development. Building evidence suggests that brain metabolic state is influenced by peripheral metabolic functions as well as by gut microbiome activity and environmental exposures where AD is seen as the failure of an integrated system.

The metabolomics approach has been widely used to identify differential metabolites including amino acids, neurotransmitters, fatty acids, lipids that can distinguish between healthy people and patients with various neurological disorders, such as AD. Previous studies have detected a panel of 20 bile acids in the rat brain, suggesting that bile acids could be playing a role in the CNS pathophysiology. Further studies on bile acid distribution in healthy C57BL/6J mice suggested that bile acids in the brains of mice are derived from circulating bile acids in blood; brain bile acids are presumably transported across the blood-brain barrier from the blood. These initial studies suggest that AD might be accompanied by abnormal metabolism of bile acids; however, these studies lack the data and analysis required to correlate effectively bile acid metabolism with various AD indicators, such as with Aβ pathology or with rates of cognitive decline. Additionally, current tools for AD diagnosis are limited to quantification of Aβ in cerebrospinal fluid (CSF), imaging via positron emission tomography (PET) and magnetic resonance imaging (MRI), as well as neurological examinations. Despite substantial progress in this field, there is no method suitable for large-scale screening of AD.

### SUMMARY

Embodiments of the present disclosure include an assay for the detection or quantification of bile acids and bile acid derivatives in a biological sample from a subject. In accordance with these embodiments, the assay includes quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample. In some embodiments, generating the bile acid profile includes calculating a ratio of the at least one bile acid derivative to the at least one bile acid, wherein the bile acid profile can be used as a basis for treating and/or diagnosing a neurological disorder.

Embodiments of the present disclosure include a method for treating a neurological disorder in a subject. In accordance with these embodiments, the method includes administering a composition comprising a bile acid modulating agent to the subject, wherein the bile acid modulating agent modulates the function of at least one of Farnesoid X Receptor (FXR) and G Protein Coupled Bile Acid Receptor 1 (GPBAR1/TGR5). In some embodiments, the administration of the composition treats the neurological disorder by ameliorating at least one symptom of the neurological disorder, wherein the at least one symptom of the neurological disorder is selected from the group consisting of: a defect in composite memory function; a defect in executive functioning; an increase in Alzheimer's Disease Assessment Scale (ADAS-Cog 13) score; an increase Spatial Pattern of Abnormality for Recognition of Early Alzheimer's disease (SPARE-AD); an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio; an increase in brain ventricular volume; an increase in brain atrophy; a decrease in brain cortical thickness; and a decrease in brain glucose metabolism. In some embodiments, the method includes quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample prior to the administration of the composition.

Embodiments of the present disclosure include a method of aiding in the determination of whether a subject has a neurological disorder. In accordance with these embodiments, the method includes quantifying levels of at least one bile acid and levels of a derivative of the at least one bile acid in a biological sample from a subject; calculating the ratio of the at least one bile acid derivative to the at least one bile acid; and determining that the subject has a neurological disorder when the ratio of the at least one bile acid derivative to the at least one bile acid exceeds 1.0.

Embodiments of the present disclosure include a biomarker panel for aiding in the determination of whether a subject has a neurological disorder. In accordance with these embodiments, the biomarker panel includes at least one primary bile acid biomarker and at least one primary bile acid derivative biomarker; wherein quantifying levels of the at least one primary bile acid biomarker and the at least one primary bile acid derivative biomarker aids in the determination of whether the a subject has a neurological disorder.

### BRIEF DESCRIPTION OF THE DRAWINGS

FIGS. 1A-1B include representative images of the cholesterol biosynthesis pathways involving primary bile acid synthesis in the liver and secondary bile acid synthesis by gut microbiome (FIG. 1A), and regulation of bile acid synthesis by feedback mechanism and bile acid transport through enterohepatic circulation (FIG. 1B).

FIGS. 2A-2G include representative graphs showing the bile acid profiles (mean concentrations) in subjects diagnosed as cognitively normal (CN), having late mild cognitive impairment (LMCI), or having Alzheimer's Disease (AD). FIG. 2A includes mean serum bile acid levels in the three diagnostic groups. FIG. 2B includes cytotoxic and neuroprotective bile acid profiles in the three diagnostic groups. FIG. 2C includes ratios of bile acids in the three diagnostic groups. Asterisks indicate statistical significance (*q<0.05, **q<0.01, and ***q<0.001). Heatmap of q-values for associations among bile acid profiles and composite scores for memory and executive functioning are shown in FIG. 2D, while FIG. 2E includes correlations with AD biomarkers, such as Tau levels. FIGS. 2F-2G include representative heatmaps and q-values for associations among bile acid profiles and CSF biomarkers, such as β-amyloid (A) and fibrillary tau (T), and brain imaging changes related to neurodegeneration (N), before (FIG. 2F) and after (FIG. 2G) adjusting for medication use. P-values estimated from linear regression adjusted and corrected for multiple testing using Benjamini-Hochberge procedure. Color code: q<0.001 (dark brown), q<0.05 (orange), q>0.05 (ivory).

FIGS. 3A-3F include representative graphs and images of the results of surface-based whole-brain analysis. A whole-brain multivariate analysis of cortical thickness was performed on a vertex-by-vertex basis to visualize the topography of the association of bile acid profiles with brain structure in an unbiased manner FIG. 3A includes representative images demonstrating that six bile acid profiles (GCDCA, GLCA, TLCA, GDCA:CA, TDCA:CA, and GLCA:CDCA) exhibited consistent patterns in their associations with CSF Aβ₁₋₄₂ or p-tau levels. The increased levels of bacterial produced secondary bile acids correlated with MRI changes and brain atrophy.

FIG. 3B includes representative images of whole-brain analysis of six bile acid profiles (GCDCA, GLCA, TLCA, GDCA:CA, TDCA:CA, and GLCA:CDCA) on brain glucose metabolism on a voxel wise level, which demonstrate significant associations with CSF Aβ₁₋₄₂ or p-tau biomarkers, FDG metabolism, and hippocampal volume. Increased levels of bacterial produced secondary bile acids correlates with glucose metabolic defects in the brain as shown by the FDG-PET imaging.

FIG. 3C includes results of whole brain analysis of the effects of APOE ε4 status on the association of bile acid profiles with brain glucose metabolism, which suggest that higher bile acid levels were associated with reduced glucose metabolism only in participants with no APOE ε4 alleles (cluster wise threshold of FDR-corrected p<0.05). FIG. 3D includes mega-analysis correlating bile acid profiles with brain imagining phenotypes; 19 bile acids were significantly associated with at least one imaging phenotype after adjusting for multiple comparisons. FIG. 3E includes results of the main effect of primary (CA), secondary (GLCA), total cytotoxic, total neuroprotective, and ratio (GCDCA:CDCA) profiles using baseline MRI scans. FIG. 3F includes results of the association of the GLCA:CDCA ratio with amyloid-β deposition measured by [¹¹C] PiB PET. The ratio (GLCA:CDCA) was significantly associated with a global mean cortical measure of Amyloid-β deposition extracted for the frontal lobe, lateral temporal lobe, parietal lobe, anterior cingulate, and precuneus. The regions with significantly increased PiB uptake in participants with increased ratio values were shown in color. The red-to-yellow scale indicates increasing statistical significance of differences in PiB uptake. Statistical maps were thresholded using a random field theory adjustment to a corrected significance level of 0.05. Two p values (p values for each vertex and p values for each cluster) are shown simultaneously. The p-value for clusters indicates significant corrected p values with the lightest blue color and the p-value for vertices indicates significant corrected p values with the lightest yellow color. Note that the p-value for vertices overlaps the p-value for clusters.

FIGS. 4A-4C include representative graphs and images of the association of serum bile acids with composite memory and executive functioning. These data demonstrate association between baseline GCDCA:CDCA ratio and longitudinal cognitive (ADAS-Cog13) and imaging (MRI: brain ventricular volume) changes during follow-up. FIG. 4A includes representative data correlating GCDCA:CDCA ratios during follow-up, and FIG. 4B includes representative data of the longitudinal cognitive (ADAS-Cog13) and composite memory score changes based on GCDCA:CDCA ratios. Lines represent trajectories on subjects on the 25th percentile (black line), 50th percentile (red line), 75th percentile (green line) of baseline GCDCA:CDCA ratio. Y-axes are ADAS-Cog13 score (left) and Ventricular Volume (right). Trajectories for these values are calculated based on the studied mixed-effects models. FIG. 4C includes analysis of the rate of cortical thinning over two and a half years and its correlation with levels of cytotoxic to neuroprotective bile acids (GCDCA:CDCA ratio).

FIGS. 5A-5B include representative graphs and images of bile acid profiles of normal mice in liver, plasma, brain and feces from the Principal component analysis (PCA) model (FIG. 5A). FIG. 5B includes representative bar graphs of total bile acids, unconjugated bile acids, conjugated bile acids, taurine conjugated bile acids, glycine conjugated bile acids, primary bile acids and secondary bile acids in liver, plasma, brain and feces of normal mice. FIG. 5C includes a representative heatmap demonstrating differences in bile acid concentrations in liver, plasma, feces and brain of normal mice were different. Values represent the Z-score values of the bile acid concentration.

FIG. 6 includes ratios of bile acids pertaining to liver and gut microbiome enzymatic activities in CN, Early MCI (EMCI), Late MCI (LMCI), and AD subjects. Three types of ratios were calculated to determine correlations with enzymatic activity changes in Alzheimer's patients. These ratios reflect one of the following: (1) Shift in bile acid metabolism from primary to alternative pathway; (2) changes in gut microbiome correlated with production of secondary bile acids; and (3) changes in glycine and taurine conjugation of secondary bile acids. Color code: Green: cognitively normal; Yellow: EMCI; Blue: LMCI; Red: AD. Composition of selected ratios stratified by clinical diagnosis. Error bars indicate standard error of the means; Asterisks indicate statistical significance (*P<E-03, **P<2E-03, and ***P<2E-05). P-values were estimated from logistic regression models and adjusted for age, sex, body mass index and APOE ε4 status. The significance level was adjusted for multiple testing according to Bonferroni method to 0.05/138=3.62E-4; LCA were excluded in the quality control steps.

FIGS. 7A-7B include a representative outline of the statistical processing and analyses conducted using the data of the present disclosure (FIG. 7A), and a list of the studied medication categories and the percentage of subjects taking these medications in each of the diagnostic categories for the ADNI-1 and ADNI-GO-2 cohorts (FIG. 7B).

### DETAILED DESCRIPTION

Embodiments of the present disclosure relate generally to the analysis of broad metabolic changes associated with neurological disorders. In particular, the present disclosure provides materials and methods relating to the use of metabolomics as a biochemical approach to identify peripheral metabolic changes and corresponding metabolic biomarkers of neurological disorders. Embodiments of the present disclosure include the use of bile acids and their derivatives as metabolic biomarkers to aid in the determination of whether a subject suffers from, or is at risk of developing, a neurological disorder, such as Alzheimer's Disease (AD).

In accordance with these embodiments, studies were conducted to investigate cholesterol metabolism and clearance through production of primary and secondary bile acids steps that take place in liver and gut and that involve human and bacterial microbiome metabolism. Bile acid induced membrane damage has been reported to positively correlate with the degree of hydrophobicity and detergent effect of each particular bile acid. The significantly increased taurine or glycine conjugated secondary bile acids were associated with and resulted from the significantly increased levels of most hydrophobic and cytotoxic DCA and LCA that are produced through intestinal microbial conversion. Recent studies have also revealed that elevated serum bile acids could act as toxic compounds to the blood brain barrier and brain and may play a pathological role in azoxymethane-induced neurological decline in mice.

Another important metabolic feature identified in the present disclosure involves significantly increased ratios of conjugated bile acids over unconjugated bile acids, such as increased GCDCA/CDCA, which correlated significantly with AD pathology and brain cortical thickness. This suggests an increase in intestinal reabsorption of conjugated bile acids by the enterocytes via the uptake transporter apical sodium-dependent bile acid transporters (ASBT), and the organic solute and steroid transporters (OST) α and β. Increased amount of conjugated bile acids in blood enter into brain, affecting brain physiology and metabolism.

Like many other steroid hormones such as neurosteroids and glucocorticoids, bile acids can act as neuroactive steroids in brain. Neurosteroids classically act to modulate GABAergic tone. Different classes of bile acids can either inhibit or potentiate N-Methyl-D-aspartate receptors (NMDARs) or gamma-aminobutyric acid receptor (GABAα) in the brain. Bile acids UDCA and CDCA have recently been shown to antagonize GABAA receptors, CDCA was shown to antagonize NMDA receptors, and overactivation of NMDARs were shown to promote neuronal death in neuropathological conditions. The activity of the NMDA receptor may increase or decrease AP in vivo and at the same time, AP pathology may drive an abnormal conformation of the NMDA receptor or deleteriously enhance the association of the NMDA receptor with certain molecules. Therefore, preservation of NMDA and GABAA receptor function by bile acid inhibition may protect against neuronal damage.

The plasma bile acid signature can be reflective of the gut environment, and a significantly altered blood bile acid profile might link altered gut microbiota to AD pathology. In the present disclosure, serum levels of 20 bile acids in control subjects (“cognitively normal” or CN), subjects with mild cognitive impairment (MCI), and subjects with AD were analyzed using a cross-sectional study design. As described herein, total bile acid levels were significantly increased in AD patients relative to controls, including six cytotoxic bile acids (GCDCA, GLCA, GDCA, TLCA, TDCA, and DCA) that were elevated from CN to MCI to AD dementia.

Additionally, increased in serum levels of secondary bile acids and increased ratios of secondary bile acids to primary bile acids (e.g., DCA/CA) were observed in AD subjects, demonstrating direct evidence of increased gut microbial conversion of bile acids, and indirect evidence for increased amount of bacteria rich in bile salt hydrolase (BSH) such as Clostridium in the intestine.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Correlated to” as used herein refers to compared to.

“Bile acid(s)” and “bile acid derivative(s),” as used herein (also abbreviated as “BA” or “Bas”) refers to any of the known bile acids and derivatives thereof, including primary and secondary bile acids, as well as any derivative of any of the known bile acids or derivatives thereof, including derivatives of a primary bile acid, a secondary bile acid, a conjugated primary acid, or a conjugated secondary bile acid. As one of skill in the art would recognize based on the present disclosure, there are many known bile acids, including but not limited to, deoxycholic acid, cholic acid, taurocholic acid, glycocholic acid, glycodeoxycholic acid, taurodeoxycholic acid, ursodeoxycholic acid and chenodeoxycholic acid, and any derivatives thereof. Each of these compounds can also be functionalized and substituted to encompass a class of compounds, which includes among other things, oxidized and reduced analogs, alkylated and acylated analogs, cyclized or bis-cyclized analogs, and analogs having a shorter or longer side chain. All of these bile acids and bile acid derivatives are included in the terms “bile acid(s)” and “bile acid derivative(s).”

“Bile acid modulating agent(s),” as used herein refers to an agent (e.g., small molecule compound, biologic molecule, aptamer, and any combinations thereof) capable of modulating the synthesis and/or processing of a bile acid or a bile acid derivative. Modulating can include both increasing activity (e.g., activation, stimulation, etc.) and decreasing (e.g., attenuation, inhibition, etc.) activity of the particular target or targets of the bile acid modulating agent, such that bile acid synthesis and/or processing is ultimately modulated. For example, a bile acid modulating agent can include agents capable of reducing the levels of secondary bile acids as compared to primary bile acids.

“Sample,” “test sample,” “specimen,” “sample from a subject,” and “patient sample” as used herein may be used interchangeable and may be a sample of blood, such as whole blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Bile Acid Synthesis, Conjugation, and Related Signaling Pathways

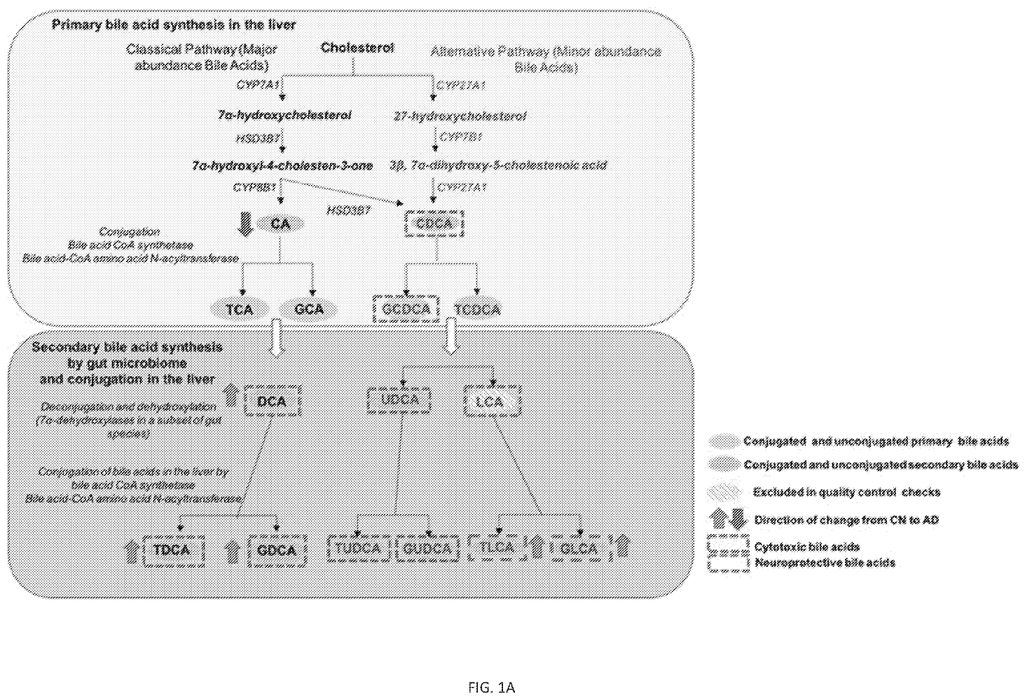

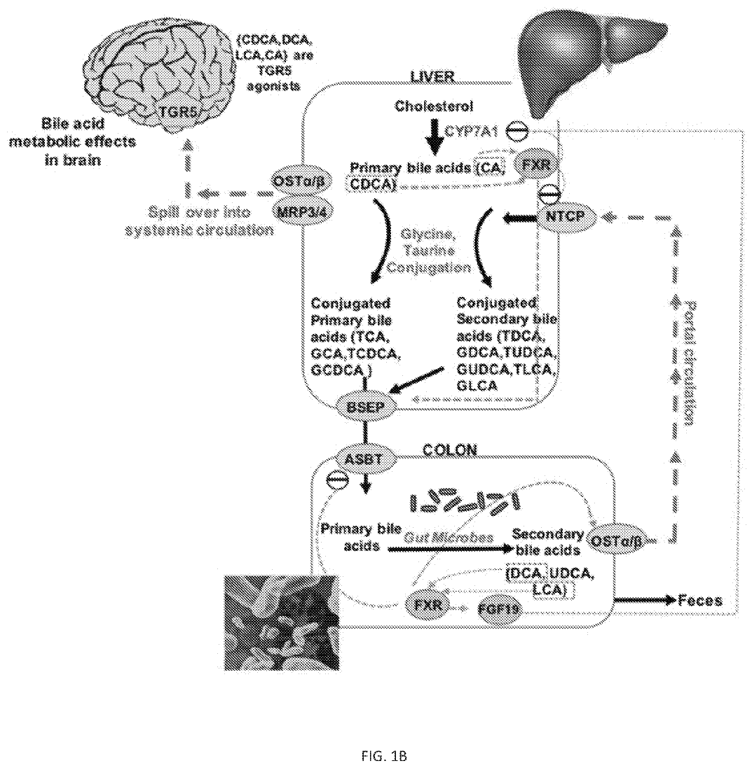

FIG. 1A includes representative images of the cholesterol biosynthesis pathways involving primary bile acid synthesis in the liver and secondary bile acid synthesis by gut microbiome. Cholesterol is cleared through production of bile acids (also referred to as “BAs”). Primary bile acids, chenodeoxycholic acid (CDCA) and cholic acid (CA), are synthesized from cholesterol, conjugated with glycine or taurine in the liver, secreted into the gallbladder via a bile salt export pump (BSEP), and transported to the intestine to be metabolized by gut bacteria (FIG. 1A). Intestinal anaerobic bacteria deconjugate the liver-derived bile acids through the action of bile salt hydrolases (BSH) to their respective free bile acids. Subsequently, anaerobe bacteria convert primary bile acids CDCA and CA into secondary bile acids lithocholic acid (LCA) and deoxycholic acid (DCA), respectively, through 7α dehydroxylation. In the terminal ileum and colon, bile acids are reabsorbed by the enterocytes and released into the portal vein for return to the liver where they are conjugated to produce their glycine and taurine forms. Bile acids are major regulators for maintaining energy homeostasis through binding to nuclear receptors, including Farnesoid A Receptor (FXR) among other receptors. They also modulate the gut microbiome; bile acid feeding in rats resulted in expansion of Clostridia populations. Secondary bile acids inhibit the pathogen Clostridium difficile in a dose-dependent manner.

FIG. 1B includes representative images of the regulation of bile acid synthesis by feedback mechanism and bile acid transport through enterohepatic circulation. In the liver, the bile acids, CDCA, DCA, LCA, and CA activate FXR, which inhibits the rate-limiting enzyme CYP7A1. The bile acids, via FXR, also inhibit the influx transporter Sodium/Taurocholate Co-transporting Polypeptide (NTCP), and induce the bile salt export pump (BSEP) and canalicular bile acid secretion. In the intestine, bile acids, via FXR, inhibit the uptake transporter apical sodium-dependent bile acid transporters (ASBT), decreasing absorption and increasing basolateral secretion into portal circulation by inducing the organic solute and steroid transporters (OST) a and β. Bile acid activated FXR in the intestine also inhibits CYP7A1 in the liver via the Fibroblast growth factor (FGF) 19 pathway. At the basolateral membrane of hepatocytes, transporters OSTα and β, and also Multidrug resistance proteins 3 and 4 (MRP3 and MRP4), secrete bile acids into systemic circulation.

The results described in the present disclosure demonstrate that the metabolic profile of bile acids is significantly modified in AD patients. In particular, bacterially produced secondary bile acids and their conjugated forms (e.g., DCA, TDCA, GDCA, TLCA, and GLCA) were progressively elevated from CN to AD. Cytotoxic bile acid levels and their ratios were increased in the various cognitive disease states and correlated with poor cognition and cognitive decline; however, no changes were observed in levels of neuroprotective bile acids. Nineteen bile acid profiles including primary (CA, CDCA, GCDCA, TMCA(a+b)), secondary (UDCA, GDCA, GLCA, GUDCA, TDCA, TLCA), cytotoxic, and ratio (DCA:CA, GDCA:DCA, TDCA:DCA, GCDCA:CDCA, TCDCA:CDCA, GLCA:CDCA, TLCA:CDCA, GUDCA:UDCA) were associated with at least one imaging phenotype after controlling for multiple testing (FDR-corrected p<0.05). Of 19 significant bile acid measures, for 3 profiles (CA, CDCA, UDCA), higher levels were associated with less structural atrophy, while for 16 profiles higher levels were associated with more structural atrophy. For one bacterially produced conjugated secondary bile acid metabolite (TLCA) and one ratio of a bacterially produced secondary bile acid metabolite to a primary bile acid metabolite (TDCA:CA), increased levels were associated with reduced cortical thickness in a widespread pattern, especially in the bilateral frontal, parietal, and temporal lobes. Lower TLCA levels were associated with increased glucose metabolism in the left temporal lobe.

Results of the present disclosure also demonstrate that altered bile acid profiles were significantly associated with structural and functional changes in brain as evidenced by more atrophy and reduced glucose metabolism in the brain. Altered bile acid profiles were also significantly associated with three CSF biomarkers, Aβ₁₋₄₂, t-tau, and p-tau. Previous studies suggested that AD patients have a marked increase in levels of CSF, t-tau, and p-tau, and a substantial reduction in CSF Aβ₁₋₄₂. Results of the present disclosure indicate that higher levels of GDCA:CA, TDCA:CA, and GLCA:CDCA were associated with decreased levels of CSF Aβ₁₋₄₂, and higher levels of GCDCA, GLCA, and TLCA were associated with increased levels of CSF t-tau and p-tau.

Results of the present disclosure also demonstrate that in AD subjects compared to CN subjects, there are significantly decreased concentrations of the primary bile acid CA, produced in the liver and increased levels of the bacterially produced, secondary bile acid DCA and its glycine and taurine derivatives. An increased ratio of DCA:CA that reflects CA 7α-dehydroxylation by gut bacteria strongly correlated with cognitive decline, a finding that was replicated in blood and brain samples of participants in the Religious Orders and Memory and Aging Project. Several genetic variants implicated in both AD and innate immunity showed associations with similar changes bile acid profiles.

### 3. Detecting or Quantifying Bile Acids

Embodiments of the present disclosure include an assay for the detection or quantification of bile acids and bile acid derivatives in a biological sample from a subject. In accordance with these embodiments, the assay can include quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample.

In some embodiments, the bile acid profile is indicative of a neurological disorder, including but not limited to of dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders. In some embodiments, the neurological disorder is Alzheimer's Disease.

In some embodiments, generating the bile acid profile comprises calculating a ratio of the at least one bile acid derivative to the at least one bile acid, as described herein. For example, the assay can include determining that the subject has a neurological disorder when the ratio of the at least one bile acid derivative to the at least one bile acid exceeds 1.0. In some embodiments, the ratio of the at least one bile acid derivative to the at least one bile acid exceeds 1.0, exceeds 1.5, exceeds 2.0, exceeds 2.5, or exceeds 5.0. In some embodiments, the ratio of the at least one bile acid derivative to the at least one bile acid is between about 1.0 and about 10.0, between about 1.0 and about 5.0, between about 1.5 and about 10.0, between about 1.5 and about 5.0, between about 2.0 and about 10.0, or between about 2.0 and about 5.0. In some embodiments, the bile acid profile is associated with at least one symptom of a neurological disorder, including but not limited to, a defect in composite memory function, a defect in executive functioning, an increase in Alzheimer's Disease Assessment Scale (ADAS-Cog 13) score, and an increase Spatial Pattern of Abnormality for Recognition of Early Alzheimer's disease (SPARE-AD). A bile acid profile may also be associated levels of proteins indicative of a neurological disorder (e.g., biomarkers), including but not limited to, an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio. In some embodiments, the bile acid profile is associated with various brain morphologies and/or functions indicative of a neurological disorder, including but not limited to, an increase in brain ventricular volume, an increase in brain atrophy, a decrease in brain cortical thickness, and a decrease in brain glucose metabolism.

In accordance with these embodiments, the assay can be used to detect and/or quantify at least one bile acid, such as cholic acid (CA), and at least one bile acid derivative, such as deoxycholic acid (DCA). The assay can be used to detect and/or quantify at least one bile acid, such as deoxycholic acid (DCA), and at least one bile acid derivative such as glucodeoxycholic acid (GDCA). The assay can be used to detect and/or quantify at least one bile acid, such as chenodeoxycholic acid (CDCA), and at least one bile acid derivative such as glycochenodeoxycholic acid (GCDCA). The assay can be used to detect and/or quantify at least one bile acid, such as chenodeoxycholic acid (CDCA), and at least one bile acid derivative, such as taurochenodeoxycholic acid (TCDCA). The assay can be used to detect and/or quantify at least one bile acid, such as chenodeoxycholic acid (CDCA), and at least one bile acid derivative such as glycolithocholic acid (GLCA). The assay can be used to detect and/or quantify at least one bile acid, such as chenodeoxycholic acid (CDCA), and at least one bile acid derivative such as taurolithocholic acid (TLCA). The assay can be used to detect and/or quantify at least one bile acid, such as ursodeoxycholic acid (UDCA), and at least one bile acid derivative such as glycoursodeoxycholine acid (GUDCA).

In some embodiments, the assay can be carried out using a biological sample, such as a biological sample from a human subject. For example, the assay may be carried out using a biological sample from the subject is at least one of whole blood, serum, plasma, and cerebral spinal fluid (CSF). In some embodiments, the assay can be performed using at least one of liquid chromatography (LC), ultra-high pressure liquid chromatography (UPLC), tandem mass spectrometry (MS), liquid chromatography tandem mass spectrometry (LC-MS-MS), and triple quadrupole tandem mass spectrometer operated in Multiple Reaction Monitoring (MRM) mode.

In some embodiments, the assay can be performed in conjunction with clinical trials to evaluate the efficacy of a therapeutic compound(s) being tested in the context of a neurological disorder. For example, bile acid profiles can be generated using the biological samples from study subjects, which can be correlated with various aspects of disease progression in order to determine whether the compounds are effective.

### 4. Methods of Treatment of Neurological Disorders

Embodiments of the present disclosure include a method for treating a neurological disorder in a subject. In accordance with these embodiments, the method can include administering a composition that includes a bile acid modulating agent to the subject. In some embodiments, the bile acid modulating agent modulates the function of at least one of Farnesoid X Receptor (FXR) and G Protein Coupled Bile Acid Receptor 1 (GPBAR1/TGR5).

In some embodiments, the method includes administering the composition to treat the neurological disorder by ameliorating at least one symptom of the neurological disorder. Symptoms of the neurological disorder can include, but are not limited to, a defect in composite memory function; a defect in executive functioning; an increase in Alzheimer's Disease Assessment Scale (ADAS-Cog 13) score; an increase Spatial Pattern of Abnormality for Recognition of Early Alzheimer's disease (SPARE-AD); an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio; an increase in brain ventricular volume; an increase in brain atrophy; a decrease in brain cortical thickness; and a decrease in brain glucose metabolism.

In some embodiments, the method of treatment includes quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample prior to the administration of the composition. In some embodiments, the bile acid profile generated can be used to evaluate treatment efficacy, and in some cases, may indicate the need for alternative treatments.

In some embodiments, the method can include treatment with bile acid modulating agents such as, but not limited to: i) FXR agonists such as Obeticholic acid, OCA, INT-747, INT-767, GW4064, GSK2324, PX-102, PX20606, GS9674, Way362362450, and fexaramine and LJN452; ii) TGR5 agonists such as INT-767, BAR502, and INT-777; iii) FGF-19 analogue, NGM-282; iv) ASBT inhibitors such as LUM-001, A4250, and GSK2330672; v) PPAR agonists such as fenofibrate, bezafibrate and GFT505; vi) UDCA-related compounds such as norUDCA and Tauroursodeoxycholate (TUDCA); vii) Fatty acid-bile acid conjugate, Aramchol; viii) Resins such as colestipol, colesevelam, colestimide, and sevelamer; ix) NTCP inhibitor, Myrcludex B; and any combinations thereof.

### 5. Methods of Diagnosis

Embodiments of the present disclosure include a method of aiding in the determination of whether a subject has a neurological disorder. In accordance with these embodiments, the method includes quantifying levels of at least one bile acid and levels of a derivative of the at least one bile acid in a biological sample from a subject, calculating the ratio of the at least one bile acid derivative to the at least one bile acid, and determining that the subject has a neurological disorder when the ratio of the at least one bile acid derivative to the at least one bile acid exceeds 1.0. In some embodiments, the ratio of the at least one bile acid derivative to the at least one bile acid exceeds 1.0, exceeds 1.5, exceeds 2.0, exceeds 2.5, or exceeds 5.0. In some embodiments, the ratio of the at least one bile acid derivative to the at least one bile acid is between about 1.0 and about 10.0, between about 1.0 and about 5.0, between about 1.5 and about 10.0, between about 1.5 and about 5.0, between about 2.0 and about 10.0, or between about 2.0 and about 5.0.

In accordance with these embodiments, the method can include detecting and/or quantifying at least one bile acid, such as cholic acid (CA), and at least one bile acid derivative, such as deoxycholic acid (DCA). The method can include detecting and/or quantifying at least one bile acid, such as deoxycholic acid (DCA), and at least one bile acid derivative such as glucodeoxycholic acid (GDCA). The method can include detecting and/or quantifying at least one bile acid, such as chenodeoxycholic acid (CDCA), and at least one bile acid derivative such as glycochenodeoxycholic acid (GCDCA). The method can include detecting and/or quantifying at least one bile acid, such as chenodeoxycholic acid (CDCA), and at least one bile acid derivative, such as taurochenodeoxycholic acid (TCDCA). The method can include detecting and/or quantifying at least one bile acid, such as chenodeoxycholic acid (CDCA), and at least one bile acid derivative such as glycolithocholic acid (GLCA). The method can include detecting and/or quantifying at least one bile acid, such as chenodeoxycholic acid (CDCA), and at least one bile acid derivative such as taurolithocholic acid (TLCA). The method can include detecting and/or quantifying at least one bile acid, such as ursodeoxycholic acid (UDCA), and at least one bile acid derivative such as glycoursodeoxycholine acid (GUDCA).

In some embodiments, the method includes aiding in the determination of whether a subject has a neurological disorder, including but not limited to, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders. In some embodiments, the neurological disorder is Alzheimer's Disease.

In some embodiments, the method is performed using a biological sample, such as a biological sample from a human subject. For example, the assay may be carried out using a biological sample from the subject is at least one of whole blood, serum, plasma, and cerebral spinal fluid (CSF). In some embodiments, the assay can be performed using at least one of liquid chromatography (LC), ultra-high pressure liquid chromatography (UPLC), tandem mass spectrometry (MS), liquid chromatography tandem mass spectrometry (LC-MS-MS), and triple quadrupole tandem mass spectrometer operated in Multiple Reaction Monitoring (MRM) mode.

In some embodiments, the method includes performing a neurological assessment of the subject to verify presence of at least one independent indicator of the neurological disorder. The neurological assessment can include at least one of a neuroimaging procedure, determining a Alzheimer's Disease Assessment Scale cognitive subscale 13 (ADAS-Cog 13) score, determining a Spatial Pattern of Abnormality for Recognition of Early Alzheimer's disease (SPARE-AD) score, measurement of executive function, measurement of memory function, measurement of brain ventricular volume, measurement of brain atrophy, measurement of cortical thickness, measurement of Amyloid β1-42 protein fragment (Aβ₁₋₄₂), measurement of total Tau (T-tau)/Aβ₁₋₄₂ ratio, and combinations thereof. In some embodiments, the independent neurological indicator correlates with levels of the at least one primary bile acid or levels of at least one primary bile acid derivative indicating the presence of the neurological disorder.

In some embodiments, the method of treatment includes quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample prior to the administration of the composition. In some embodiments, the bile acid profile generated can be used to evaluate treatment efficacy, and in some cases, may indicate the need for alternative treatments.

### 6. Bile Acid Biomarkers and Panels

Embodiments of the present disclosure include a biomarker panel for aiding in the determination of whether a subject has a neurological disorder. In accordance with these embodiments, the biomarker panel can include at least one primary bile acid biomarker and at least one primary bile acid derivative biomarker. Quantifying levels of the at least one primary bile acid biomarker and the at least one primary bile acid derivative biomarker can aid in the determination of whether the subject has a neurological disorder.

In some embodiments, the at least one primary bile acid biomarker is selected from cholic acid (CA) and chenodeoxycholic acid (CDCA). In some embodiments, the at least one primary bile acid derivative biomarker is selected from deoxycholic acid (DCA), glycodeoxycholic acid (GDCA), and glycochenodeoxycholic acid (GCDCA).

In some embodiments, the biomarker panel further includes a biomarker for a gene and/or protein, including but not limited to ABI3, CLU, CR1, EPHA1, INPP5D, MEF2C, MS4A6A, PLCG2, TREM2, CYP7A1, IMPA2, LRRC7, CYCS, GPC6, FOXN3, and CNTNAP4 and any combinations thereof.

### 7. Materials and Methods

a. Subjects and Samples

Neuroimaging and baseline metabolomics data used in the present disclosure were acquired from the Alzheimer's Disease Neuroimaging Initiative (ADNI) database (adni.loni.usc.edu). ADNI-1 was launched in 2004 by the National Institute on Aging (NIA), the National Institute of Biomedical Imaging and Bioengineering, the Food and Drug Administration, private pharmaceutical companies and non-profit organizations (Weiner et al., 2015). ADNI-1, followed by ADNI-GO and ADNI-2, has recruited over 1500 adults, ages 55 to 90, to participate in the research, consisting of cognitively normal older individuals, people with early and late mild cognitive impairment (EMCI and LMCI, respectively), and Alzheimer's disease (AD). AD dementia diagnosis was established based on the NINDS-ADRDA criteria for probable AD, whereas MCI subjects did not meet these AD criteria and had largely intact general cognition and functional performance and met predetermined criteria for amnestic MCI (Weiner et al., 2015). ADNI subject underwent extensive clinical and cognitive testing, including the Alzheimer's Disease Assessment Scale-Cognition (ADAS-Cog13), which was used as a measure of general cognition in this analysis (www.adni-info.org).

Two sample sets of elderly subjects were analyzed with neuroimaging and metabolomics data from the ADNI-1 and ADNI-GO-2 cohorts. ADNI-GO and ADNI-2 subjects were referred to as “ADNI-GO-2,” and the data collection was identical for them both. All ADNI studies are conducted according to the Good Clinical Practice guidelines, the Declaration of Helsinki, and U.S. 21 CFR Part 50 (Protection of Human Subjects), and Part 56 (Institutional Review Boards). Written informed consent was obtained from all participants before protocol-specific procedures were performed.

The following data, fully or partially, were available for the subjects: Demographics data including age, gender, education and body mass index; Fasting serum bile acid measurements; Cognitive assessments including ADAS-Cog 13, memory and executive function composite scores; CSF Aβ₁₋₄₂, total tau (t-tau) and tau phosphorylated at threonine 181 (p-tau₁₈₁) markers; Clinical diagnosis at baseline through 36-month follow-up; and Medication data. All the subjects were seen in six visits, including screening, baseline and four follow-up visits at 6, 12, 24, and 36 months and assessed for cognitive and everyday functioning. Clinical and demographic variables for the subjects are summarized in Table 1 (below). Clinical and demographic variables for a larger pool of the ADNI subjects which includes the subjects in Table 1 are summarized in Table 9 (below).

TABLE 1
Demographics of ANDI study subjects stratified by baseline diagnosis^{a}.

| | | CN | EMCI | LMCI | AD | |
|---|---|---|---|---|---|---|
| Variable | N | (N = 370) | (N = 284) | (N = 505) | (N = 305) | p-value^{b} |
| Age | 1464 | 74.58(5.71) | 71.12(7.51) | 73.95(7.59) | 74.70(7.79) | P < 0.001 |
| Sex: Female, No. (%) | 1464 | 190(51%) | 130(46%) | (139)39% | (139)46% | P = 0.004 |
| Education, years | 1464 | 16.28(2.92) | 15.95(2.66) | 15.87(2.90) | 15.16(3.00) | P < 0.001 |
| BAD (Kg/M²) | 1461 | 27.05(4.46) | 28.06(5.41) | 26.54(4.25) | 25.83(4.69) | P < 0.001 |
| ≥1 APOE ε4 allele, | 1464 | 104(28%) | 121(43%) | 273(54%) | 202(66%) | P < 0.001 |
| No. (%) | | | | | | |
| ADAS-Cog13^{c} | 1455 | 9.19(4.17) | 12.64(5.40) | 18.67(6.62) | 29.67(8.20) | P < 0.001 |
| Statin use: Yes, No. (%) | 1464 | 42%(156) | 48%(137) | 50%(250) | 47%(144) | P = 0.18 |

^{a}Data are reported as mean(SD) unless otherwise indicated. Bolded values indicate statistical signficance. SD: Standard deviation.
^{b}Based on 2-sample t tests, or Pearson χ² tests.
^{c}Score explanations: ADAS-Cog13 range, 0 (best) to 85 (worst).
Abbreviations: AD: Alzheimer's disease; BMI: Body mass index; CN: Cognitively normal; EMCI: Early mild cognitive impairment; LMCI: Late mild cognitive impairment; ADAS-Cog13, Alzheimer Disease Assessment Scale 13-item cognitive subscale.

TABLE 9
Demographic characteristics of ADNI participants by baseline diagnosis

| | | CN | SMC | EMCI | LMCI | AD |
|---|---|---|---|---|---|---|
| Variable | N | (N = 370) | (N = 98) | (N = 284) | (N = 505) | (N = 305) |
| Age | 1562 | 74.58(5.71) | 72.18(5.63) | 71.12(7.51) | 73.95(7.59) | 74.70(7.79) |
| Female, No. (%) | 1562 | 190(51) | 56(57) | 130(46) | 139(39) | 139(46) |
| Education, years | 1562 | 16.28(2.92) | 16.71(2.56) | 15.95(2.66) | 15.87(2.90) | 15.16(3.00) |
| BMI (kg/M²) | 1558 | 27.05(4.46) | 28.22(6.24) | 28.06(5.41) | 26.54(4.25) | 25.83(4.69) |
| APOE ε4 status (+ %) | 1562 | 104(28) | 32(33) | 121(43) | 273(54) | 202(66) |

b. ANDI-1 Baseline Samples

Bile acid profile, neuroimaging and clinical data for all the ADNI-1 sub were downloaded from the ADNI Image Data Archive on January 2016. The dataset includes 833 samples with unique identifiers belonging to 801 subjects. There were duplicate aliquots from the same CSF draw for 24 subjects to help us evaluate analytical performance.

The ADNI-1 cohort included three diagnostic groups: subjects with AD, late MCI, and healthy elderly (cognitively normal) participants. A series of data-preprocessing steps were applied to the metabolite profile to remove non-fasting participants, multivariate outliers, and normalized the metabolite values. After the quality control steps, the final dataset comprised 721 individuals (average age±s.d.=75.03±6.83 years; 414 men/307 women) including 172 AD, 356 LMCI, and 193 healthy participants. All samples profiled were in fasting state.

c. ANDI-GO-2 Baseline Samples

The bile acid profile for ADNI-GO-2 was downloaded on January 2017. The dataset contains 904 samples belonging to 874 participants. Two additional clinical diagnostic groups were introduced in ADNI-GO-2 protocol. Healthy controls were subdivided into CN participants without significant memory concerns and control participants with significant memory concerns (SMC). Also included subjects with MCI who were further subdivided into early and late MCI (EMCI, LMCI). The preprocessed ADNI-GO-2 dataset includes 840 individuals (average age s.d.=72.40±7.31 years; 434 men/406 women) including 177 healthy control without significant memory concern, 99 SMC, 283 EMCI, 148 LMCI, and 133 AD.

d. Religious Orders Study (ROS) and Rush Memory and Aging Project (ROSMAP) Samples

The Religious Orders Study (ROS) and the Memory and Aging Project (MAP) are both longitudinal cohort studies of aging and AD run from Rush University. ROS enrolled individuals from more than 40 groups of religious orders (nuns, priests, brothers) across the United States for longitudinal clinical analysis. MAP was designed to complement the ROS study by using a similar structure and design as ROS, but enrolling participants with a wider range of life experiences and socioeconomic status. The entire ROSMAP cohort consists of approximately 3000 participants. A subset of serum bile acids in 566 subjects (446 elderly cognitively normal individuals, 109 mild cognitive impairment and 11 AD patients) was measured, as well as a subset of bile acids in brain samples of 100 subjects. Clinical and demographic variables for the subjects are summarized in Table 2 (below).

TABLE 2
Demographics of ROSMAP participants stratified by clinical diagnosis^{a}.

| | Subjects with Blood Samples | | | |
|---|---|---|---|---|
| | | CN | MCI | AD |
| | N | (N = 446) | (N = 109) | (N = 11) |
| Age, years | 566 | 80.77(7.37) | 86.49(6.00) | 84.42(8.58) |
| Sex: Female, No(%) | 566 | 21%( 93) | 23%( 25) | 27%(3) |
| Education, years | 566 | 15.79(3.12) | 15.26(2.95) | 17.00(4.10) |
| ≥1 APOE ε4 allele, No. (%) | 477 | 18%( 68) | 25%( 23) | 44%(4) |
| Global Cognition^{b} | 564 | 0.39(0.44) | −0.42(0.46) | −1.19(0.84) |

^{a}Data are reported as mean(SD) unless otherwise indicated.
^{b}Raw scores from a battery of cognitive tests were converted to Z scores and averaged to yield a global cognitive function summary. Mean and standard deviation at baseline were used to compute the z-scores.
A negative z-score means that someone has an overall score that is lower than the average of the entire cohort at baseline.

e. Rotterdam Study (RS)

The Rotterdam Study (RS) cohort was used to investigate associations among bile acids with AD genetic variants. RS is a prospective population based study. At the baseline examination in 1990-93, 7983 subjects≥55 years of age were recruited from the Ommoord district of Rotterdam (RS-D. All the study participants were extensively interviewed and physically examined at baseline and after every 3 to 4 years. During 2000 to 2001, the baseline cohort (RS-I) was expanded with 3011 subjects≥55 years of age, who were not yet part of RS-I (RS-II). In a second expansion of RS, 3932 persons with age≥45 years were recruited during 2006-2008 (RS-III). The study has been approved by the Medical Ethical Committee of Erasmus Medical Center and by the Ministry of Health, Welfare and Sport of the Netherlands. Written Informed consents were also obtained from each study participant to participate and to collect information from their treating physicians. Clinical and demographic variables for the subjects are summarized in Table 3 (below).

TABLE 3
Demographics of Rotterdam Study cohort.

| | | Dementia free subjects |
|---|---|---|
| Variable | N | at time of blood sample |
| Age, Years | 488 | 73.1(6.3) |
| Sex: Female, No(%) | 488 | 53% |
| Body Mass Index, kg/m2 | 488 | 26.7(3.7) |
| ≥1 APOE ε4 allele, No. (%) | 488 | 26% |

^{a}Data are reported as mean(SD) unless otherwise indicated.

f. Quantification of Serum Bile Acids

Bile acid quantification was performed by the Duke Proteomics and Metabolomics Shared Resource by liquid chromatography tandem mass spectrometry (LC-MS-MS) using the Biocrates Life Sciences Bile Acids Kit (BIOCRATES Life Science AG, Innsbruck, Austria) according to the manufacturer's instructions. Sample analysis of bile acids are performed by a UPLC (ultra-high pressure liquid chromatography) tandem mass spectrometry (MS) method using a reversed phase analytical column for analyte separation (LC/MS-MS). Selective analyte detection is accomplished by use of a triple quadrupole tandem mass spectrometer operated in Multiple Reaction Monitoring (MRM) mode, in which specific precursor to product ion transitions are measured for every analyte and stable isotope labeled internal standard. The Biocrates bile acids assay quantifies bile acids, 16 of which are normally detected in human serum (Table 4).

TABLE 4
Measured Bile Acids.

| Abbreviation | Bile Acid Name | Primary | Conjugated |
|---|---|---|---|
| CA | Cholic acid | x | |
| CDCA | Chenodeoxycholic acid | x | |
| GCA | Glycocholic acid | x | x |
| GCDCA | Glycochenodeoxycholic acid | x | x |
| TCA | Taurocholic acid | x | x |
| TCDCA | Taurochenodeoxycholic acid | x | x |
| MCA(a) | Muricholic acid (alpha) | x | |
| MCA(b) | Muricholic acid (beta) | x | |
| MCA(o) | Muricholic acid (omega) | x | |
| TMCA(a + b) | Tauromuricholic acid (alpha + beta) | x | x |
| DCA | Deoxycholic acid | x | |
| HDCA | Hyodeoxycholic acid | | |
| LCA | Lithocholic acid | | |
| UDCA | Ursodeoxycholic acid | | |
| GDCA | Glycodeoxycholic acid | | x |
| TDCA | Taurodeoxycholic acid | | x |
| GLCA | Glycolithocholic acid | | x |
| GLCAS | Glycolithocholic acid sulphate | | x |
| TLCA | Taurolithocholic acid | | x |
| TLCAS | Taurolithocholic acid sulphate | | x |
| GUDCA | Glycoursodeoxycholic acid | | x |
| TUDCA | Tauroursodeoxycholic acid | | x |

De-identified morning serum samples were analyzed by following the manufacturer's protocol, with metabolomics labs blinded to diagnosis and pathological data. To assess the precision of the measured analytes, a set of blinded analytical replicates (24 pairs in ADNI-1 and 15 triples in ADNI-GO-2) were supplied by ADNI; keeping the Metabolomics Consortium blinded of the matching pairs. Following unbinding the bile acid profiles, the accuracy was measured using intra-class correlation coefficient (ICC) and coefficient of variation (CV) using the blinded replicates.

Bile acid measurements in 566 participants of the ROS/MAP studies was carried using ultra-performance liquid chromatography coupled to a tandem mass spectrometry (UPLC-MS/MS) system (ACQUITY UPLC-Xevo TQ-S, Waters Corp., Milford, Mass.). A subset of bile acids were measured and used to replicate ADNI findings. In the RS study, bile acids were measured in 488 participants using the non-targeted Metabolon platform (Durham, N.C., USA). For a subset of 100 ROS/MAP study subjects, both brain and blood samples were available. Briefly, extracts of brain samples along with bile acid reference standards were subjected to instrumental analysis. A Waters ACQUITY ultra performance LC system coupled with a Waters XEVO TQ-S mass spectrometer with an ESI source controlled by MassLynx 4.1 software (Waters, Milford, Mass.) was used for all analyses. Chromatographic separations were performed with an ACQUITY BEH C18 column (1.7 μm, 100 mm×2.1 mm internal dimensions) (Waters, Milford, Mass.). UPLC-MS raw data obtained with negative mode were analyzed using TargetLynx applications manager version 4.1 (Waters Corp., Milford, Mass.) to obtain calibration equations and the quantitative concentration of each bile acid in the samples.

g. Quality Control of Bile Acid Profiles

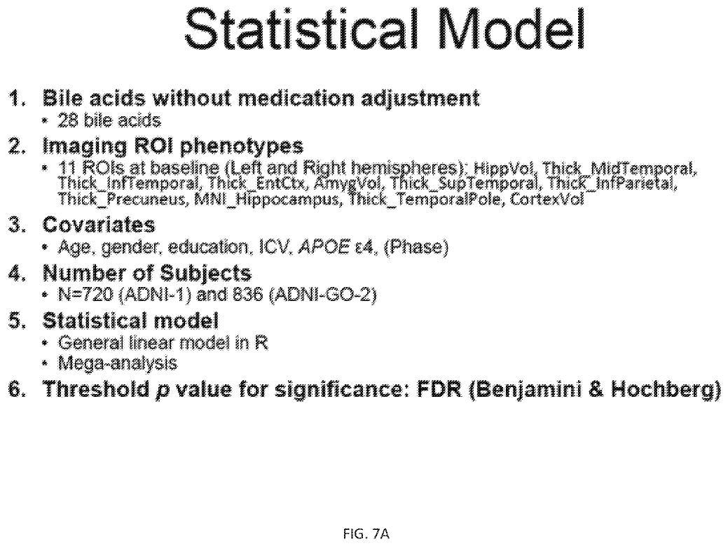

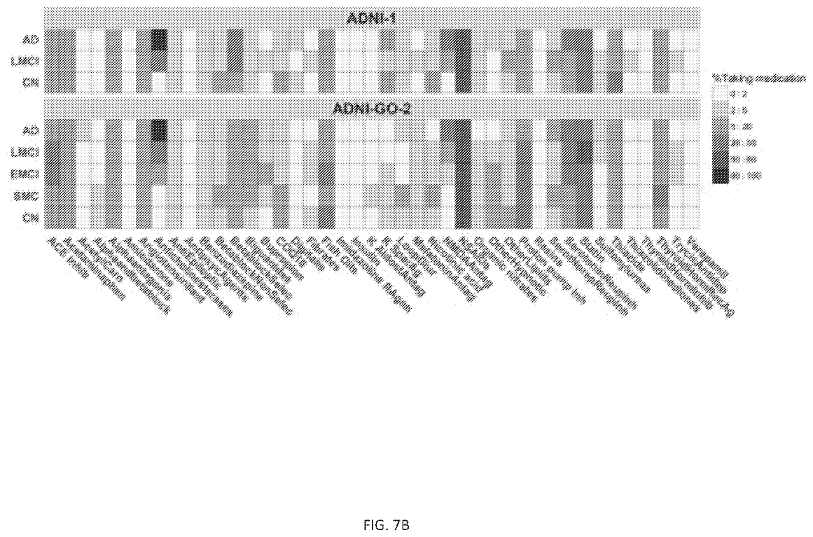

All statistical processing and analyses were conducted using the open source, statistical software, R v3.2.4. The preprocessing included the steps briefly described herein, and graphically depicted as a flowchart in FIG. 7A. In summary, two sulphates (GLCAS and TLCAS) were excluded from dataset as Biocrates has not analytically validated the results for these compounds. Two exclusion criteria were applied to filter individual metabolites as part of the data quality control (QC) evaluation process based on the blinded replicates: 1) coefficient of variation (CV)>25% across plates; and 2) intraclass correlation coefficient (ICC)<0.65. Additionally, metabolites with >40% of measurements below the lower limit of detection (<LOD) were excluded from the analysis. Combined, these three steps allow only the most robust metabolites from the panel to be used for subsequent analyses, and reduced the total number of metabolites reported in the dataset from 22 metabolites to 15 metabolites in study subject cohorts (Table 5). LOD values were imputed using minimum value divided by 2 for each specific metabolite. Also, subjects identified as non-fasting and subjects lacking corresponding body mass index (BMI) were determined to be pre-analytical outliers, were flagged and removed from the dataset.

TABLE 5
Coefficient of Variation (CV) and Infra Class Correlation Coefficient (ICC)
calculated based on the replicated samples on different plates and result of
the quality control (QC) process in ADNI.

| | ADNI | | | |
|---|---|---|---|---|
| Metabolite | % <LOD | CV (%) | ICC | QC Result |
| CA | 1.84 | 7.02 | 0.99 | Passed |
| CDCA | 7.49 | 12.55 | 0.99 | Passed |
| DCA | 1.23 | 6.96 | 0.99 | Passed |
| GCA | 0.12 | 7.34 | 0.98 | Passed |
| GCDCA | 0 | 6.96 | 0.98 | Passed |
| GDCA | 1.35 | 6.53 | 0.98 | Passed |
| GLCA | 32.15 | 9.06 | 0.98 | Passed |
| GLCAS | | | | Excluded |
| GUDCA | 4.66 | 9.73 | 0.97 | Passed |
| HDCA | 96.93 | NA | NA | Failed |
| LCA | 36.81 | 49.5 | 0.28 | Failed |
| TCA | 35.95 | 6.82 | 0.97 | Passed |
| TCDCA | 2.58 | 6.25 | 0.98 | Passed |
| TDCA | 2.7 | 7.92 | 0.97 | Passed |
| TLCA | 25.89 | 18.1 | 0.89 | Passed |
| TLCAS | | | | Excluded |
| MCA (a, b, c) | 100 | NA | NA | Failed |
| TMCA | 28.22 | 15.07 | 0.97 | Passed |
| TUDCA | 5.521 | 21.49 | 0.89 | Passed |
| UDCA | 39.75 | 13.27 | 0.96 | Passed |

The last steps in the statistical quality control pipeline serve to combine replicate measurements to give one value per biological sample, filter out any statistical outlier subjects, and perform log-transformation if necessary. For the participants with duplicated or triplicated measurements, average values of the two measured values were used in further analyses. The data were checked for outlier subjects by performing principal components analysis and evaluating the subject distance from the centroid in the K-dimensional space based on principal components that explained >90% cumulative variance. Subjects with distances>7 SD from the mean were flagged as outliers. Finally, log₁₀ transformation was performed for those metabolites which show p-value for D'Agostino p<0.05 and skewness>2.

h. Medication Adjustment and Composite Metabolite Scores

The normalized preprocessed obtained from the QC step was used for subsequent association analyses directly or was adjusted to take into account the effect of medications on the levels of the bile acids. For classification of medications and approaches to adjust for their effects on metabolism see Appendix IV in the ADNI database; medication data are available for 42 major medication classes used to treat psychiatric (including different categories of benzodiazepines, antipsychotics, and antidepressants) and cardiovascular conditions (including different categories of anti-hypertensives, cholesterol treatment, and anti-diabetics), as well as dietary supplements (Co-Q10, fish oil, nicotinic acid, and acetyl L-carnitine). Medication used for each category were systematically coded and available for model-based evaluations of the influence of each drug type on metabolite levels. Intake of any medication within a category was coded as present or absent. Dose effect was not evaluated. The list of the studied medication categories and the percentage of subjects taking these medications in each of the diagnostic categories for the ADNI-1 and ADNI-GO-2 cohorts is listed in FIG. 7B. For each metabolite, medications were backward-selected via Bayesian information criteria (BIC) to select an optimal combination of medications for preventing confounding while limiting model complexity. Two AD medication classes (i.e., anticholinesterases and NMDA receptor antagonist) were excluded from this process due to highly collinearity (Cramer's V=0.65 and 0.52) of these medications with diagnosis. We refer to the residuals of each metabolite as the medication adjusted metabolites. The residuals then carried forward to test associations with clinical outcomes.

Finally, the medication adjusted and unadjusted datasets were extended by 11 ratios of bile acids, the total bile acids, cytotoxic bile acids as summation of DCA, GCDCA, GLCA, GDCA, TLCA, and TDCA and neuroprotective bile acids as summation of UDCA, GCDCA, TUDCA, and CDCA resulting in a total of 29 metabolic variables.

i. CSF Biomarkers and Laboratory Measures

Lumbar puncture was performed in the mornings after an overnight fast. Aβ₁₋₄₂, t-tau, and p-tau₁₈₁ were measured using the multiplex xMAP Luminex platform (Luminex Corp, Austin, Tex.) with Innogenetics immunoassay kit-based reagents (INNO-BIA AlzBio3; Ghent, Belgium; for research use-only reagents) (Kang et al., 2015). CSF samples were available and measured for 48.18% of the CN, 51.68% of the LMCI and 55.81% of the AD subjects in ADNI1. In ADNI-GO-2, CSF measurements of 84.7% of the CN, 90.1% SMC, 88.33% of the EMCI, 93.91% LMCI and 86.46% of the AD are available. These CSF biomarkers were also analyzed in pristine aliquots of 2,401 ADNI CSF samples using automated Roche Elecsys electrochemiluminescence immunoassays (same reagent lot for each of these three biomarkers). Linear regression analysis was performed using age, sex, study phase (ADNI-1 or ADNI-GO/2), and APOE ε4 status as covariates, followed by FDR-based multiple comparison adjustment with the Benjamini-Hochberg procedure.

j. Cognitive and Clinical Stage Measures

The ADNI participants underwent a comprehensive cognitive and clinical battery. The modified Alzheimer's Disease Assessment Scale-cognition sub-scale (ADAS-Cog 13; range, 0 [best] to 85 [worst] points; Mohs et al., 1997) was used as overall indices of general cognitive performance, which were collected on all subjects at the screening visit and subsequent follow-up visits. Categorical response variables included clinical diagnosis at baseline and MCI conversion (MCI-NonConverter, MCI-Converter). For the ROS/MAP cohort, cognition was measured with a battery of 21 cognitive tests, 19 of which are used to measure five domains of cognitive performance. Raw scores from each cognitive tests were converted to Z scores and averaged to create a global cognitive performance variable. Mean and standard deviation at baseline were used to compute z-scores. A negative z-score means that an individual has an overall score that is lower than the average of the entire sample at baseline.

In addition to ADAS-Cog13, two cognitive composite scores were employed: executive function (Trail Making Test Part B [TMT-B], a composite executive function score) and memory (Weschler Memory Scale—Revised Logical Memory Immediate and Delayed, a composite memory score). The scores were developed using a detailed neuropsychological assessment including measures of memory and executive function and modem psychometric theory applied to item-level data from the ADNI neuropsychological battery (Crane et al., 2008; Crane et al., 2012; Gibbons et al., 2012).

k. MRI Measures

T1-weighted brain MRI scans at baseline were acquired using a sagittal 3D MP-RAGE sequence following the ADNI MRI protocol. As detailed in previous studies (Jack et al., 2010; Jack et al., 2008), two widely employed automated MRI analysis techniques were independently used to process MRI scans and extract whole brain-wide and ROI (region of interest)-based neuroimaging endophenotypes (grey matter (GM) density, volume, and cortical thickness): whole-brain voxel-based morphometry (VBM) using statistical parametric mapping 8 (SPM8) and FreeSurfer V5.1 (Risacher et al., 2013; Nho et al., 2013; Nho et al., 2015). In particular, FreeSurfer was used to extract brain-wide cortical thickness by automated segmentation and parcellation. The cortical surface was reconstructed to measure thickness at each vertex on surface. The cortical thickness was calculated by taking the Euclidean distance between the grey/white boundary and the grey/cerebrospinal fluid (CSF) boundary at each vertex on surface (Fischl et al., 1999; Dale et al., 1999; Chung et al., 2010).

l. Regional Analysis of Structural MRI

Mean values (volume, cortical thickness, grey matter density) of 11 AD-related brain regions of interest (ROIs) were used as phenotypes (hippocampus volume, middle temporal cortical thickness, inferior temporal cortical thickness, amygdale volume, superior temporal cortical thickness, inferior parietal cortical thickness, precunneus cortical thickness, hippocampus GM density, mean temporal pole cortical thickness, and cerebral cortex GM volume). A linear regression approach was performed using age at baseline, gender, years of education, the number of APOE ε4 alleles, and intracranial volume (ICV) as covariates. For a mega-analysis, a dummy variable (ADNI-1=0 and ADNI-GO-2=1) was used as additional covariate. FDR (false discovery rate)-based multiple comparison adjustment with the Benjamini-Hochberg procedure was used because phenotypes were strongly correlated each other.

m. Regional Analysis of Structural MRI and FDG PET

Mean ICV-adjusted hippocampal volume was used as an MRI-related phenotype. Linear regression was performed using age, sex, years of education, intracranial volume (ICV), magnetic field strength, and APOE ε4 status as covariates. For FDG PET, a mean SUVR value was extracted from a global cortical ROI representing regions where AD subjects show decreased glucose metabolism relative to cognitively normal older participants (CN) from the full ADNI-1 cohort, normalized to pons. Linear regression analysis was performed using age, sex, protocol under which individuals were recruited (ADNI-1 or ADNI-GO/2), and APOE ε4 status as covariates. FDR (false discovery rate)-based multiple comparison adjustment with the Benjamini-Hochberg procedure was used because the AD biomarker phenotypes were strongly correlated with each other. Not accounting for this high collinearity of dependent variables would lead to an overly stringent correction for multiple testing.

n. Unbiased Whole Brain Imaging Analysis

The SurfStat software package (www.math.mcgill.ca/keith/surfstat/) was used to perform a multivariate analysis of cortical thickness and to examine the effect of bile acid profiles on brain structural changes on vertex-by-vertex bases by applying a general linear model (GLM) approach. GLMs were developed using age at baseline, gender, years of education, the number of APOE ε4 allele, and intracranial volume (ICV) as covariates. Change rate estimates were calculated using cortical thickness for each vertex from baseline and 24-month scans for each participant. A GLM was used to assess correlations of MRI change rate estimates with bile acid profiles. Age at baseline, gender, education, the number of APOE ε4 alleles, and baseline total cerebral cortex GM volume were included as covariates. For a mega-analysis, a dummy variable (ADNI-1=0 and ADNI-GO-2=1) was used as additional covariate. In the whole brain surface-based analysis, the adjustment for multiple comparisons was performed using the random field theory correction method at a 0.05 level of significance (Hagler et al., 2006; Hayasaka et al., 2004). Processed FDG PET images were used to perform a voxel-wise statistical analysis of the effect of bile acid levels on brain glucose metabolism across the whole brain using SPM8. Linear regression analysis was performed using age, sex, and protocol under which individuals were recruited (ADNI-1 or ADNI-GO/2) as covariates. In the voxel wise whole brain analysis, the significant statistical parameters were selected to correspond to a cluster-wise threshold of p<0.05 (FDR-corrected).

o. Processing of [¹¹C] PiB-PET Scans and Association Analysis

Pre-processed PiB PET scans (co-registered, averaged, standardized image and voxel size, uniform resolution) were downloaded from the LONI ADNI site (www.adni.loni.usc.edu/) as described in previously reports for detailed methods for acquisition and processing of PiB-PET scans for the ADNI sample Jagust et al., 2010; Jagust et al., 2009). The mean SUVR (standardized uptake value ratio) value of normalized PiB uptake values from five ROIs (anterior cingulate, frontal cortex, lateral temporal, parietal cortex, and precuneus) was used as a quantitative phenotype for the association analysis in an initial analysis. The processed PiB-PET images were then used to perform a voxel- and cluster-wise statistical analysis of the effect of bile acid levels on amyloid burden across the whole brain by SPM8. A linear regression analysis was performed using age and gender as covariates. In addition, the number of APOE ε4 allele was included as a covariate to investigate the effect of the APOE ε4 allele on the significance. The Benjamini-Hochberg FDR-based multiple comparison adjustment was used. The statistical parametric map of the regression t-statistics was analyzed in SPM8 using a voxel-level significance threshold of p<0.001 (uncorrected) and minimum cluster size k=50 contiguous voxels.

p. Statistical Analysis

Differences of demographic, clinical, cognitive and CSF markers between the clinical diagnostic groups were evaluated using the nonparametric Kruskal-Wallis test (for continuous variables) and Pearson Chi-square test (for categorical variables). The exact same statistical analyses were done for ADNI-1, ADNI-GO-2 cohorts and the dataset obtained by merging the ADNI-1 and ADNI-GO-2 cohorts. The results of the analyses of the merged dataset are referred to as mega-analysis results.

To determine whether the bile acid levels are associated with the outcomes of interest (i.e., diagnosis, ADAS-Cog13, composite memory and executive function scores, CSF markers), univariate association analysis of the 15 individual bile acids and 14 composite metabolite scores (a total of 29 simultaneous hypothesis tests per outcome) was performed with each outcome for both the medication adjusted and unadjusted datasets. Two sets of covariates were provided to each statistical model: a set of forced-in covariates, and a set of model selectable covariates. Stepwise backward selection (with BIC) was employed to eliminate nonsignificant selectable covariates from the regression models. The p-values obtained from association analyses were adjusted to account for multiple testing using the Benjamin and Hochberg false discovery rate (FDR) procedure. The adjusted p-values depicted as q-values were considered significant at the 5% level.

A multinomial logistic regression was used to examine the relationship between bile acid measures and the prevalence of LMCI and AD. For this analyses, the SMC and EMCI participants were reduced from the ADNI-GO-2 and the merged dataset to have the exact same clinical diagnostic groups in both cohorts (three-level dependent variables: cognitively normal, LMCI, and AD). The significance of the metabolites was tested using Wald's test. The following covariates were used in both the medication adjusted and unadjusted bile acids: age, education (in years) and cohort name as the forced-in covariates and gender, number of copies of minor alleles of APOE ε4 variant and log₁₀ body mass index (BMI) as the model selectable variables.

The cross-sectional association of cognitive measurements (ADAS-Cog 13, memory and executive functions) and CSF biomarkers (Aβ₁₋₄₂, t-tau, p-tau and t-tau/Aβ₁₋₄₂) with the bile acids was assessed using a linear regression model with the aforementioned sets of forced-in and selectable covariates, with the exception of the cognitive measurements where the education was added as an extra forced covariate.

A Cox hazard model including age, gender, APOE ε4 presence, and education as covariates was used to evaluate the association of metabolite levels with progression from MCI to AD with a median follow-up of 2.1 years (IQR: 1.5-3.3). A mixed-effects model that included age, gender, education, APOE ε4 presence, time, and metabolite level as independent variables was used to study longitudinal associations between the metabolites and cognitive changes (ADAS-Cog13 and memory and executive composite scores) during follow-up in the MCI participants (AD participants were excluded due to short follow-up) (Pinheiro and Bates, 2000). All the models accounted for baseline cognitive measures for each participant.

For each metabolite, separate binary logistic regression models were conducted to examine its cross-sectional association with baseline diagnosis as dependent variable (e.g., AD vs. CN, AD vs. EMCI, AD vs. LMCI, LMCI vs. CN and EMCI vs. CN). Additionally, logistic regression models were performed to compare bile acid levels between the MCI-NonConverter and MCI-Converter groups. Cox proportional hazard models were used to evaluate the association of metabolite levels with progression from MCI (combined EMCI and LMCI subjects) to AD. The cross-sectional association of ADAS-Cog13 with bile acids was assessed using separate linear regression models with square root of ADS-Cog13 as the dependent variable and models were adjusted for years of education as well as the aforementioned sets of covariates.

In ROS/MAP studies, the most recent sample for each individual was retained (1 sample per individual). Linear regression models with Global Cognition score as dependent variable and metabolites as independent variables were used to assess the association of serum and brain bile acids with cognition while adjusting for sex, age, copies of APOE ε4, and years of education.

q. Genetic Association Study and Locus Annotation

Whole-genome sequencing (WGS) was performed on blood-derived genomic DNA samples obtained from 817 ADNI participants. Samples were sequenced on the Illumina HiSeq2000. For data processing and QC, an established analysis pipeline based on GATK was used. Briefly, raw sequencing data from Illumina qseq files were converted into FASTQ files that were then subjected to quality checks and read statistics using FastQC. Short-read sequences were mapped to the NCBI reference human genome (build 37) using BWA-mem. During the alignment, potential PCR duplicates were removed. After completing initial alignment, the alignment was further refined by locally realigning any suspicious reads. The reported base calling quality scores obtained from the sequencer were re-calibrated to account for covariates of base errors such as sequencing technology and machine cycle. Finally, the realigned reads were written to a BAM file for further analysis. The analysis-ready BAM files were analyzed to identify all variants with statistical evidence for an alternate allele present among samples using GATK HaplotypeCaller for multi-sample variant callings. The quality of the variant calls was assessed by comparing sequencing-derived SNPs with those obtained from the Illumina Omni 2.5M genotyping array in the same individuals in order to estimate the concordance rate for each individual. All subject had a mean concordance rate of 99.9%. Since population stratification is known to cause spurious association in disease studies, the analyses was restricted to only non-Hispanic Caucasian participants that clustered with CEU (Utah residents with Northern and Western European ancestry from the CEPH collection)+TSI (Toscani in Italia) populations using HapMap 3 genotype data and the multidimensional scaling (MDS) analysis. Associations of AD risk variants in immune-related genes with selected metabolic traits were computed using sex, age, and body mass index (BMI) as covariates.

DNA genotyping in the participants of the RS cohort was performed using 550K, 550K duo, or 610K Illumina arrays at the internal genotyping facility of Erasmus Medical Center, Rotterdam. Study samples with excess autosomal heterozygosity, call rate<97.5%, ethnic outliers and duplicate or family relationships were excluded during quality control analysis. Genotype exclusion criteria further included call rate<95%, Hardy-Weinberg equilibrium p<1.0×10⁻⁶ and Minor Allele Frequency (MAF)<1%. Genetic variants were imputed to the Haplotype Reference Consortium (HRC) reference panel (version 1.0) using the Michigan imputation server. The server uses SHAPEIT2 (v2.r790) to phase genotype data and performs imputation with the Minimac 3 software. Genotyping and imputation information was available for all participants included in the current study. As in the ADNI, metabolic trait associations for AD risk variants were adjusted for sex, age, and BMI.

Reference genetic associations with bile acid profiles in healthy individuals were obtained from supplementary data of the atlas of genetic influences on blood metabolites (Ref. No. 24816252). Lookup of genetic associations of candidate AD genes involved in innate immunity was done with no threshold of significance for single bile acid levels, and a threshold of P<1.0×10⁻⁴ for the DCA:CA ratio. To obtain genetic associations of DCA, only suggestive significant results were included with P<1.0×10⁻⁵. Gene and complex trait annotations of the 13 resulting genetic loci were performed using the SNiPA tool v3.2 (Ref. No. 25431330) and the NHGRI-EBI Catalog of published genome-wide association studies (available at: www.ebi.ac.uk/gwas; accessed 02/01/2018, version 1.0) (Ref. No. 27899670). Lookup of genetic associations for DCA candidate variants was performed using the IGAP repository (Ref. No. 24162737).

### 8. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Cross-Sectional Association of Bile Acids with Clinical Diagnosis, and Cognitive and CNS Measurements

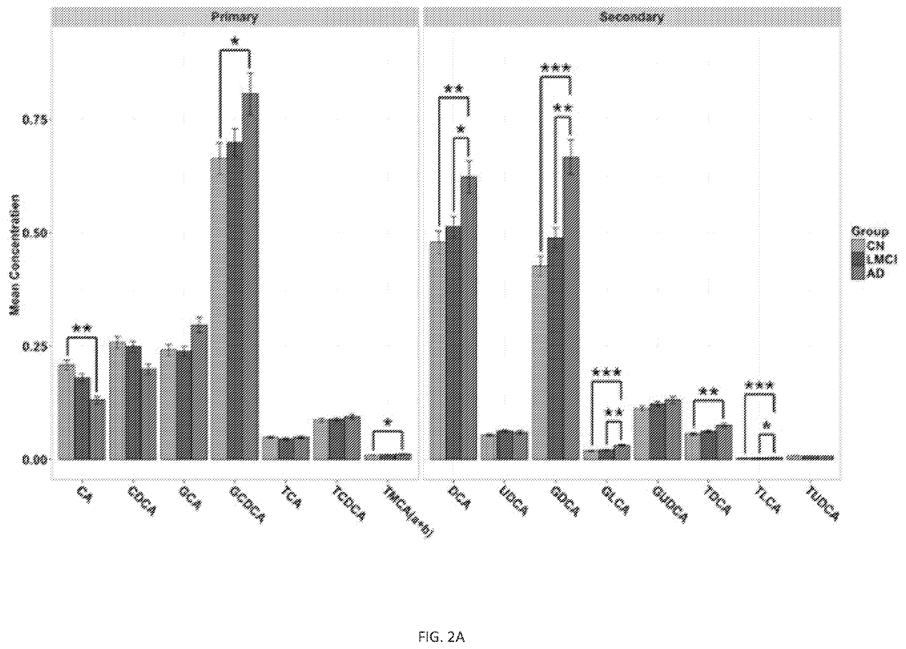

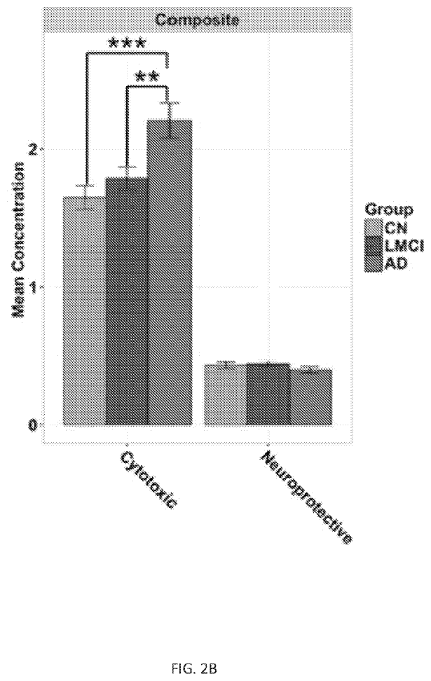

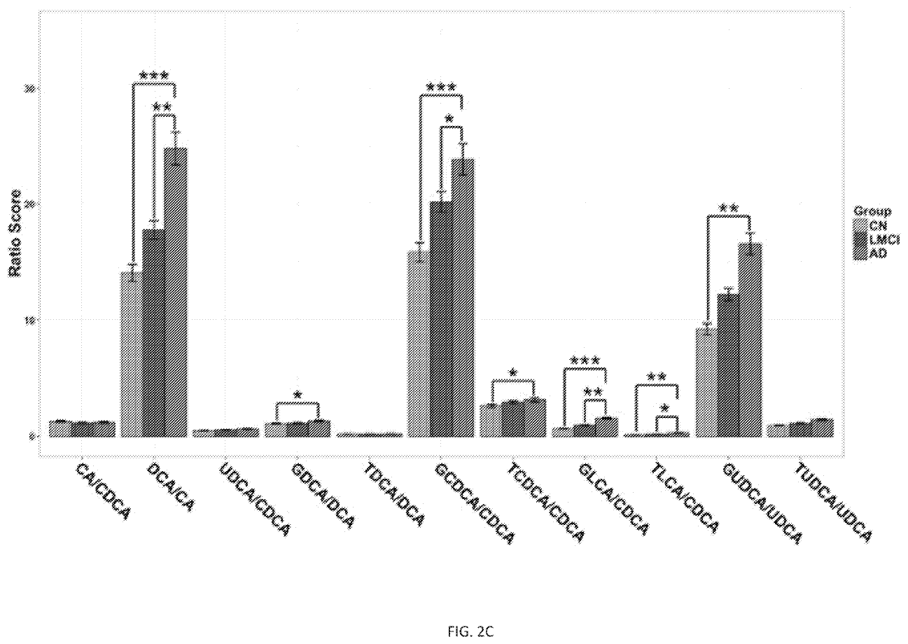

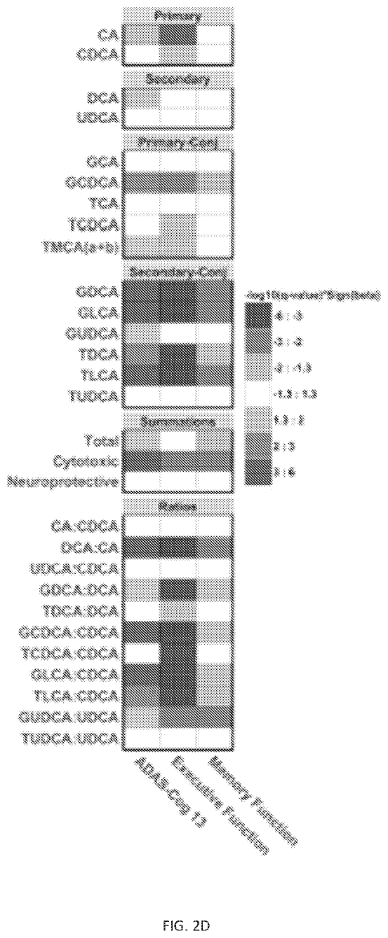

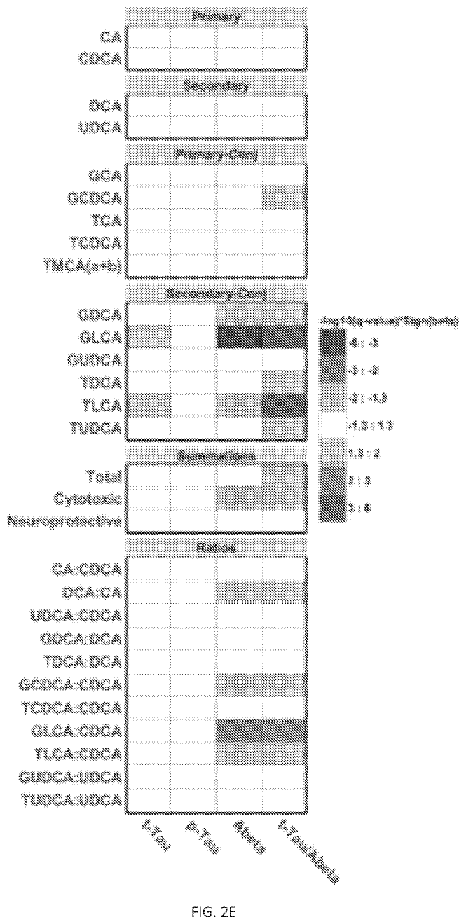

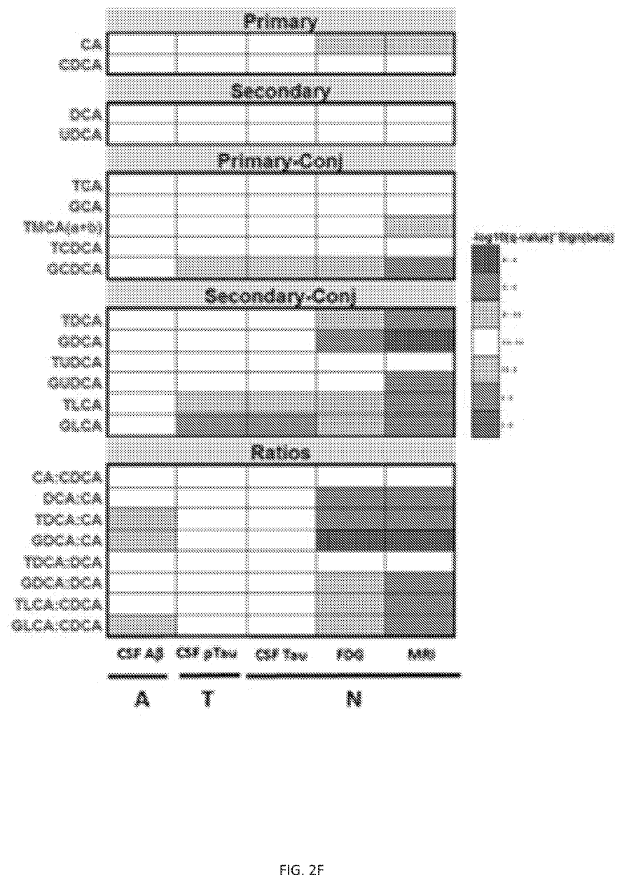

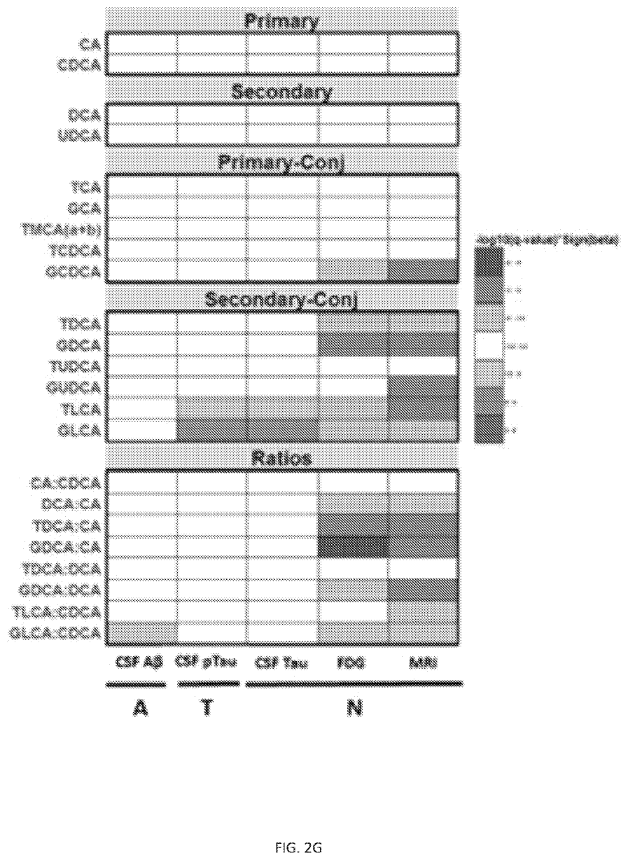

The results disclosed herein are based on the combined ADNI-1 and ADNI-GO-2 datasets referred to as mega-analysis. (Separate analysis of ADNI-1 and GO/2 can be provided upon request.) Mean total bile acids increased from CN to LMCI to AD in both cohorts and many secondary conjugated bile acids which are cytotoxic were mainly increased (FIG. 2A). The primary bile acid CA was the only one decreased and primary bile acid GCDCA was increased. TMCA, a previously described murine bile acid, was present in our study subjects and increased in disease state. Cytotoxic bile acids were generally dysregulated during the development of AD. A significant increase in the odds of diagnosed AD rather than CN as the mean cytotoxic bile acids increases was observed (OR=2.68, q-value=1.46*10⁻⁴). Comparing LMCI and CN groups, higher levels of cytotoxic bile acids were correlated with higher prevalence of LMCI (OR=1.99, q-value=7.16*10⁻³). No significant differences among the diagnostic groups were found for neuroprotective bile acids (FIG. 2B).

Comparisons between the AD and CN groups revealed a significant decrease in mean concentration of CA (OR=0.62, q-value 1.46*10⁻³), while higher levels of two primary bile acids (GCDCA and TMC (a+b)) and 5 secondary bile acids (DCA, GDCA, TDCA, GLCA, and TLCA) were significantly associated with higher odds of a diagnosis of AD. Between the AD and LMCI groups, higher values of four secondary bile acids (DCA, GDCA, GLCA, and TLCA) were significantly associated with higher prevalence of AD (FIG. 2A). No significant differences were observed between CN and LMCI groups although trends indicating increased cytotoxic bile acids in the LMCI were observed. Increases in the ratio of conjugated cytotoxic over neuroprotective bile acids (DCA:CA, GDCA:DCA, GCDCA:CDCA, TCDCA:CDCA, GLCA:CDCA, TLCA:CDCA, and GCDCA:CDCA) significantly increased the odds of an AD diagnosis (FIG. 2C).

The association between ADAS-Cog13 and composite scores for memory and executive functioning and bile acid profiles was evaluated. While CA was negatively associated with ADAS-Cog13 score, 6 other bile acid metabolites (bacterially produced secondary bile acid DCA and 5 conjugated cytotoxic bile acids, GCDCA, GDCA, GLCA, TDCA, and TLCA) were positively associated with ADAS-Cog13 (FIG. 2D). Similar to ADS-Cog13 results, secondary bacterially produced bile acid DCA and 4 conjugated cytotoxic bile acids (GCDCA, GDCA, GLCA, TDCA, and TLCA) were also negatively associated with composite memory and executive functioning. Two primary bile acids, CA and CDCA, were positively correlated with executive function (FIG. 2D).

In concordance with the diagnosis and cognition analyses, bile acids were also significantly correlated with CSF biomarkers. Conjugated cytotoxic bile acids GLCA and TLCA were significantly associated with t-tau, Aβ₁₋₄₂ and t-tau/Aβ₁₋₄₂ ratio. In addition, four conjugated bile acids (GCDCA, GDCA, TDCA, TUDCA) were significantly associated with Aβ₁₋₄₂ or/and t-tau/Aβ₁₋₄₂ ratio. Four ratios of conjugated cytotoxic bile acids to neuroprotective bile acids were significantly associated with Aβ₁₋₄₂ or/and t-tau/Aβ₁₋₄₂ (FIG. 2E).

Biomarkers of β-amyloid (“A”). As shown in FIGS. 2F-2G, CSF Aβ₁₋₄₂ levels were used as a biomarker of β-amyloid. Bile acid profiles were evaluated for associations with CSF Aβ₁₋₄₂ biomarker by performing an association analysis for 15 bile acid metabolites and 8 relevant ratios with APOE ε4 status as a covariate. These selected ratios reflected enzymatic dysfunctions in liver and changes in gut microbiome metabolism. As shown in FIG. 2F, after applying FDR-based multiple comparison correction, three bile acid ratios were identified as significantly associated with CSF Aβ₁₋₄₂ levels. Regression coefficients of the three bile acid ratios of bacterially produced conjugated secondary bile acids to primary bile acids (GDCA:CA, TDCA:CA, and GLCA:CDCA) showed negative associations, indicating higher levels were associated with lower CSF Aβ₁₋₄₂ values (i.e., CSF Aβ₁₋₄₂ positivity). Lower CSF Aβ₁₋₄₂ levels have been shown to be indicative of AD.

Biomarkers of fibrillary tau (“T”). As shown in FIGS. 2F-2G, CSF phosphorylated tau (p-tau) levels were used as the biomarker of fibrillary tau. Associations among 23 bile acids and relevant ratios with the CSF p-tau biomarker were investigated, including the use of APOE ε4 status as a covariate. Three significant associations were identified after controlling for multiple testing using FDR (corrected p<0.05) (FIG. 2E). For one conjugated primary bile acid metabolite (GCDCA), higher GCDCA levels were associated with higher CSF p-tau values. For two bacterially produced conjugated secondary bile acid metabolites (GLCA and TLCA), higher levels were correlated with higher CSF p-tau values. Higher CSF p-tau levels have been shown to be indicative of AD.

Biomarkers of neurodegeneration (“N”). As shown in FIGS. 2F-2G, structural atrophy on MRI, FDG PET metabolism, and CSF total tau (t-tau) levels were used as biomarkers of neurodegeneration or neuronal injury.

Structural atrophy was measured using MRI analysis (FIGS. 2F-2G). Associations among bile acid metabolites and bile acid ratios with mean hippocampal volume were investigated, using APOE ε4 status as a covariate. Among 23 bile characteristics, 14 bile acid ratios were significantly associated with hippocampal volume after controlling for multiple testing using FDR (FIG. 2F; p<0.05). For one primary bile acid metabolite, lower CA levels were associated with decreased hippocampal volume. However, for two conjugated primary bile acid metabolites (GCDCA and TMCA(a+b)) and five bacterially produced conjugated secondary bile acid metabolites (GDCA, GLCA, GUDCA, TDCA, and TLCA), higher levels were associated with decreased hippocampal volume. In addition, higher levels of six ratios of bacterially produced secondary bile acid metabolite to primary bile acid metabolite (DCA:CA, GDCA:CA, TDCA:CA, GDCA:DCA, GLCA:CDCA, and TLCA:CDCA) were associated with decreased hippocampal volume. Reductions in hippocampal volume have been shown to be indicative of AD.

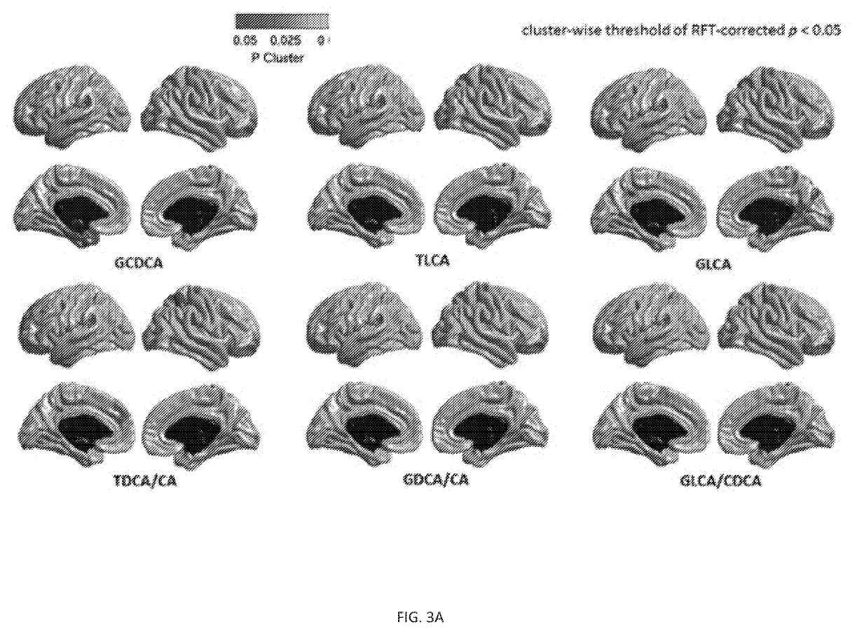

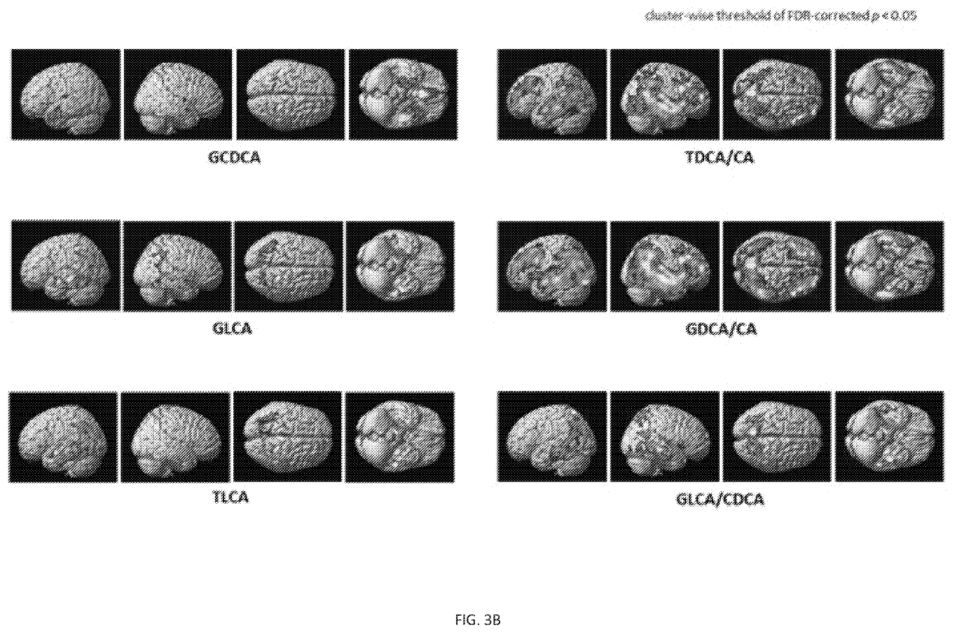

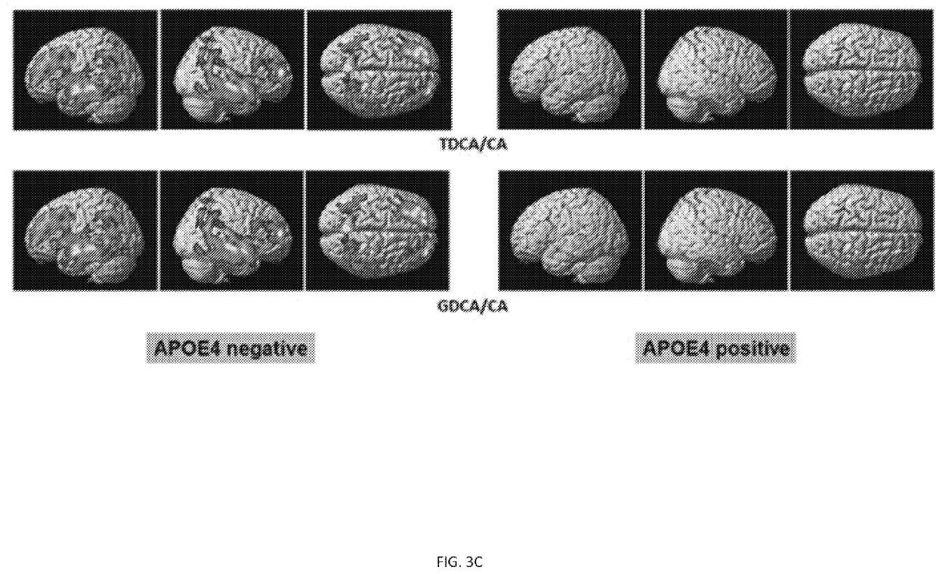

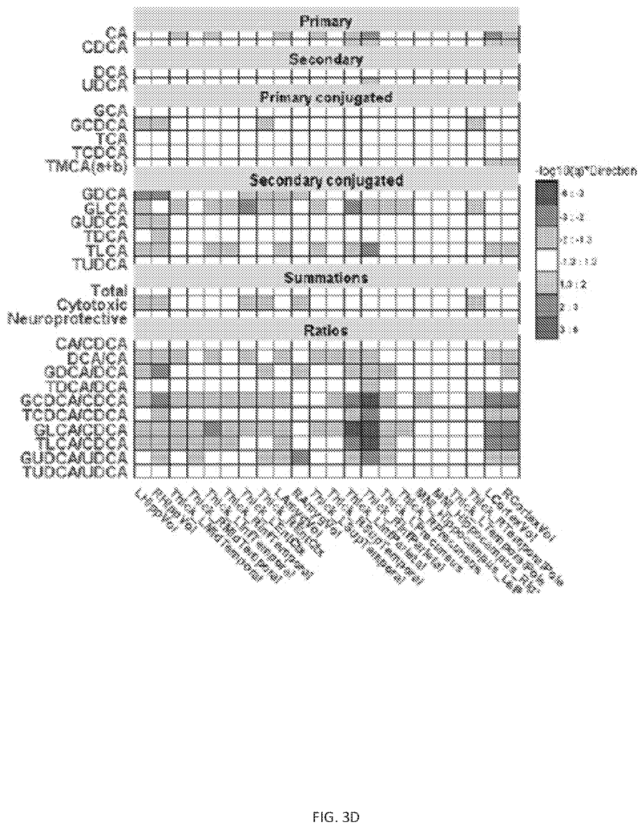

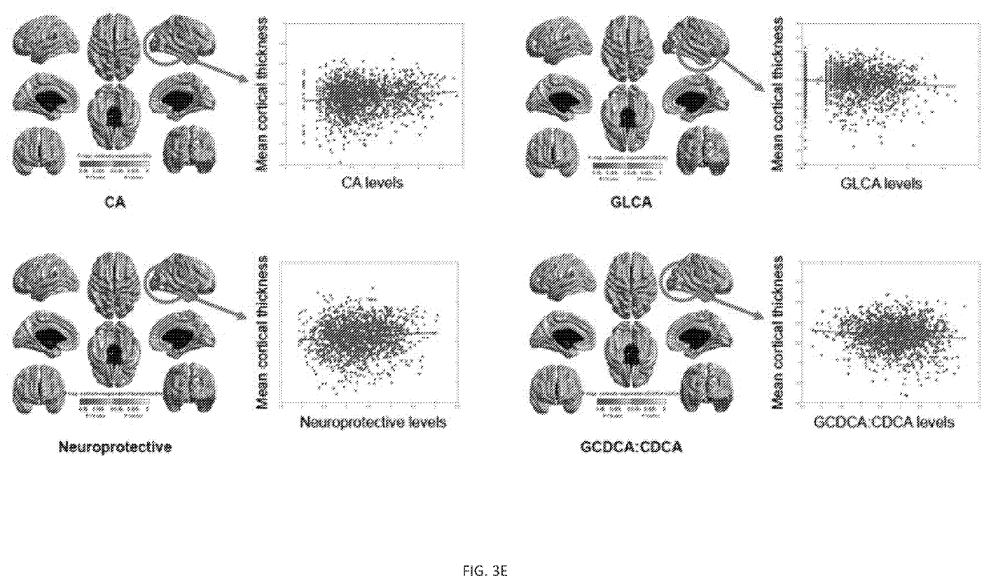

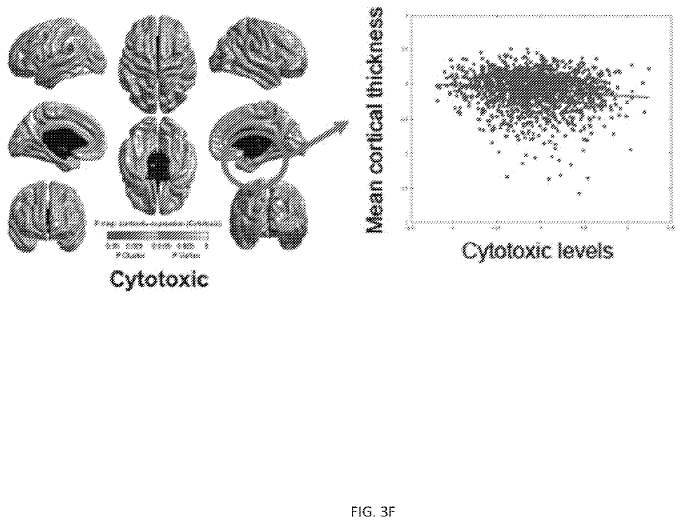

A detailed whole-brain surface-based analysis using multivariate regression models was performed to investigate associations among bile acids on whole-brain cortical thickness. Six bile profiles were significantly associated with CSF Aβ₁₋₄₂ biomarker (“A”) or CSF p-tau biomarker (“T”). Significant associations were identified for all six bile profiles (cluster wise threshold of RFT-corrected p<0.05), which showed consistent patterns in the associations of CSF Aβ₁₋₄₂ or p-tau levels (FIG. 3A). Higher levels of a conjugated primary bile acid (GCDCA) were significantly associated with reduced cortical thickness especially in bilateral entorhinal cortices. Increased levels of one bacterially produced conjugated secondary bile acid metabolite (GLCA) and two ratios of bacterially produced secondary bile acid metabolites to primary bile metabolites (GDCA:CA and GLCA:CDCA) were significantly associated with reduced cortical thickness in the bilateral frontal, parietal, and temporal lobes including the entorhinal cortex. For one bacterially produced conjugated secondary bile acid metabolite (TLCA) and one ratio of a bacterially produced secondary bile acid metabolite to a primary bile acid metabolite (TDCA:CA), increased levels were associated with reduced cortical thickness in a widespread pattern, especially in the bilateral frontal, parietal, and temporal lobes.

Blood glucose metabolism was measured using FDG Positron Emission Tomography (PET). Association analysis was performed for 23 bile acids and ratios with global cortical glucose metabolism measured by FDG PET scans across 1,066 participants with both FDG PET scans and bile acid measurements. The association testing including APOE ε4 status as a covariate, identified 12 bile acid characteristics significantly associated with brain glucose metabolism after controlling for multiple testing using FDR (FIGS. 2F-2G) (p<0.05). For one primary bile acid metabolite, lower CA levels were associated with reduced glucose metabolism. In contrast, for one conjugated primary bile acid metabolite (GCDCA), four bacterially produced conjugated secondary bile acid metabolites (GDCA, GLCA, TDCA, and TLCA), and six ratios of bacterially produced secondary bile acid metabolites to primary bile acid metabolites (DCA:CA, GDCA:CA, TDCA:CA, GDCA:DCA, GLCA:CDCA, and TLCA:CDCA), higher bile acid ratio levels were associated with reduced glucose metabolism.

A detailed whole-brain analysis was also performed to determine the effects of bile acids on brain glucose metabolism on a voxel wise level for six bile acids and ratios (GCDCA, GLCA, TLCA, GDCA:CA, TDCA:CA, and GLCA:CDCA) that were significantly associated with CSF Aβ₁₋₄₂ or p-tau biomarkers, FDG metabolism, and hippocampal volume. Significant associations for all six bile acid profiles were demonstrated (cluster wise threshold of FDR-corrected p<0.05), which showed consistent patterns in the associations of CSF Aβ₁₋₄₂ or p-tau levels (FIG. 3B). Higher levels of a conjugated primary bile acid GCDCA were significantly associated with reduced glucose metabolism especially in the bilateral hippocampi, which showed consistent patterns with the associations of cortical thickness. Increased levels of one bacterially produced conjugated secondary bile acid metabolite (GLCA) and one ratio of a bacterially produced secondary bile metabolite to a primary bile metabolite (GLCA:CDCA) were significantly associated with reduced glucose metabolism in the bilateral temporal and parietal lobes. Lower TLCA levels, a bacterially produced conjugated secondary bile acid metabolite, were associated with increased glucose metabolism in the left temporal lobe. For two ratios (GDCA:CA and TDCA:CA) of bacterially produced secondary bile acid metabolite to a primary bile acid metabolite, higher ratio levels were significantly associated with reduced glucose metabolism in a widespread pattern, especially in the bilateral frontal, parietal, and temporal lobes.

Whole brain analysis was also performed to investigate the effects of APOE ε4 status on the association of the six bile profiles with brain glucose metabolism. There was evidence that higher bile acid levels were associated with reduced glucose metabolism only in participants with no APOE ε4 alleles (cluster wise threshold of FDR-corrected p<0.05) (FIG. 3C). The associations between one ratio (GDCA:CA) of a bacterially produced secondary bile acid metabolite to a primary bile acid metabolite and glucose metabolism was statistically significant only in subjects with no APOE ε4 allele in a widespread pattern, but not in those with one or more APOE ε4 alleles. Of note, for TDCA:CA, only participants with no APOE ε4 allele showed significant associations of metabolic changes with glucose metabolism in the bilateral temporal, parietal, and frontal lobes.

Experiments were also conducted to evaluate whether 23 bile acids and ratios were associated with the CSF t-tau biomarker including APOE ε4 status as a covariate. Three significant associations were identified after controlling for multiple testing using FDR (corrected p<0.05) (FIGS. 2F-2G). Higher levels of GCDCA, a conjugated primary bile acid metabolite, and GLCA and TLCA, bacterially produced secondary bile acid metabolites, were associated with higher CSF t-tau values.

Medication use was explored as a potential confounder (FIG. 2G). Overall, the primary associations described above remained significant after adjustment for medication use, with minor exceptions. CA and TMCA(a+b), after adjustment for medication use, were no longer significantly associated with neurodegeneration as measured by MRI. In addition, GCDCA was no longer associated with p-tau or t-tau, and GDCA:CA and TDCA:CA were no longer associated with amyloid-β after adjustment for medications. In addition, GCDCA was no longer associated with p-tau or t-tau, and GDCA:CA and TDCA:CA were no longer associated with amyloid-β after adjustment for medications.

### Example 2

### Cross-Sectional Association of Bile Acids with Brain Structure

The association of bile acid profiles with AD-related structural MRI-based ROI mean values of volumes, cortical thicknesses, and GM densities was investigated. In the ADNI-1 group, eight bile acids were identified as being significantly associated with at least one imaging phenotype and a ratio (e.g., GCDCA:CDCA); this finding was replicated in an independent ADNI-GO-2 cohort. Furthermore, mega-analysis, used to increase detection power by combining two independent cohorts, demonstrated 19 bile acids were significantly associated with at least one imaging phenotype after adjusting for multiple comparisons (FIG. 3D). For two primary bile acids (CA and CDCA), higher bile acid levels were associated with less structural atrophy, while for other two primary conjugated bile acids (GCDCA and TMCA (a+b)), higher levels were associated with more structural atrophy. For the secondary bile acid UDCA, and five conjugated secondary bile acids (GDCA, GLCA, GUDCA, TDCA, and TLCA), total cytotoxic profile and 8 ratio profiles (DCA:CA, GDCA:DCA, TDCA:DCA, GCDCA:CDCA, TCDCA:CDCA, GLCA:CDCA, GUDCA:UDCA, and TLCA:CDCA) were associated with a greater amount of structural atrophy (FIG. 3D). Following this ROI-based analysis, a detailed whole brain surface-based analysis was performed using multivariate regression models to assess the effect of bile acid profiles on whole-brain cortical thickness. FIG. 3E includes results of the main effect of primary (CA), secondary (GLCA), total cytotoxic, total neuroprotective, and ratio (GCDCA:CDCA) profiles using baseline MRI scans. For the primary bile acid profiles (CA and CDCA), highly significant clusters were found in the bilateral temporal and parietal lobes, where the cortical thickness is associated with bile acid levels in the positive direction. For the secondary bile acid profiles (GLCD and TLCD), higher levels were associated with reduced cortical thickness especially in the bilateral temporal, frontal, parietal lobes. Increased sum of cytotoxic bile acid profiles was associated with reduced cortical thickness in the bilateral medial temporal lobes including entorhinal cortex. Increased sum of neuroprotective bile acid profiles was associated with large cortical thickness in the bilateral parietal lobes. A widespread pattern of significant vertices was also detected in the association analysis of ratios of cytotoxic to neuroprotective bile acid profiles with cortical thickness. Increased ratios (DCA:CA, GDCA:DCA, GCDCA:CDCA, TCDCA:CDCA, GLCA:CDCA, GUDCA:UDCA, and TLCA:CDCA) were significantly associated with reduced cortical thickness especially in the bilateral frontal, parietal, and temporal lobes.

### Example 3

Cross-Sectional Association of Bile Acids with Amyloid-β Load Measured by [¹¹C] PiB PET

The association of bile acid profiles with a global mean cortical measure of Amyloid-β deposition extracted for the frontal lobe, lateral temporal lobe, parietal lobe, anterior cingulate, and precuneus was investigated in the ADNI-1 cohort (FIG. 3F). A ratio (GLCA/CDCA) was significantly associated with cortical amyloid-β levels after a multiple comparison adjustment, where increased ratio values were correlated with higher cortical amyloid-β levels. Following this analysis, in the ADNI-1 cohort, an unbiased whole brain analysis was performed to determine the effect of the ratio (GLCA/CDCA) on a voxel- and cluster-wise level. Higher mean PiB uptake in participants with increased ratio values was found in a widespread pattern, especially bilateral frontal, parietal, and temporal lobes (p<0.001, uncorrected; k=50 voxels). In the independent ADNI-GO-2 cohort, the significant associations in the left temporal lobe were replicated.

### Example 4

### Metabolites Associated with Longitudinal Outcomes

Using four year longitudinal data available for the ADNI participants, levels of metabolites at baseline were evaluated for whether they were associated with 1) ADAS-Cog13 changes; and 2) memory composite score using mixed effects models. Progression from MCI to dementia was assessed by applying a Cox hazards model for the available follow-up. For these analyses, only the ratios that were cross-sectionally associated with clinical diagnosis, cognitive scores, CSF markers and imaging phenotypes were selected (GCDCA:CDCA, GLCA:CDCA and TLCA:CDCA). After adjusting for FDR, only GCDCA:CDCA showed a positive association with cognitive decline (13=0.055, q-value=0.024). All the three ratios showed an inverse association with the composite memory score. No significant association was observed for conversion from MCI to AD.

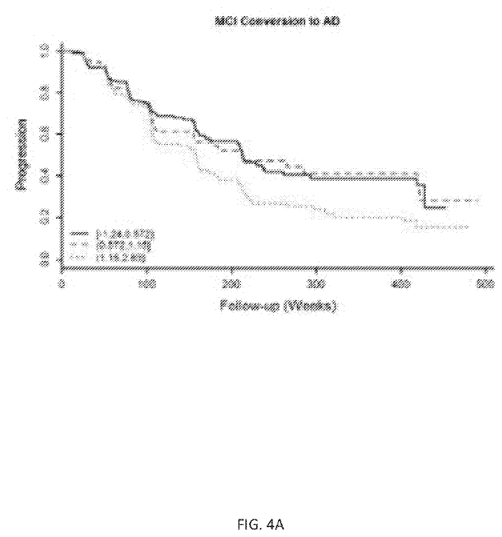

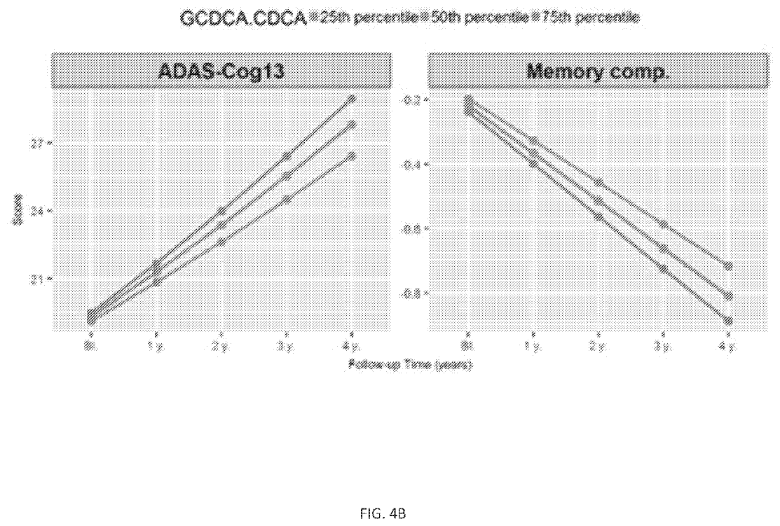

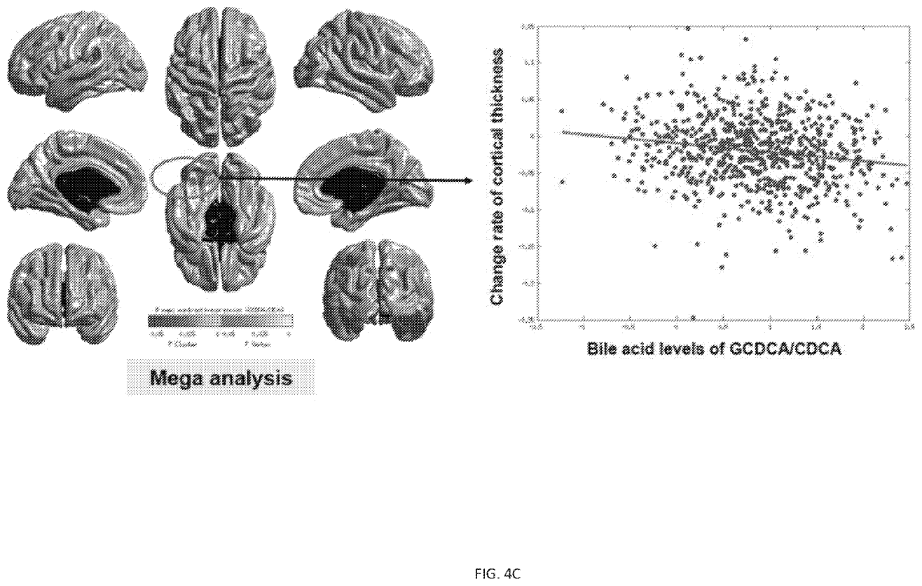

The data represented in FIGS. 4A-4B demonstrate the association between GCDCA:CDCA (presented as tertiles) and longitudinal cognitive (ADAS-Cog13) and composite memory score change. An unbiased whole brain surface-based analysis using longitudinal change rate of cortical thickness over two years after baseline identified a significant cluster in the left temporal and occipital lobes for a ratio profile (GCDCA:CDCA; FIG. 4C), where high levels were associated with small changes in rate of cortical thickness.

### Example 5

### Bile Acid Profiles in Mouse Models

Ultra-performance liquid chromatography-triple quadruple mass spectrometry (UPLC-TQMS, Waters, XEVO G2-S) was used to quantitatively measure the bile acid levels in plasma, liver, brain and feces in normal male C57BL/6J mice aged week 8 (n=6). All separations were performed with an ACQUITY BEH C18 column (1.7 μM, 100 mm×2.1 mm internal dimensions) (Waters, Milford, Mass.). Data acquisition was performed using MassLynx version 4.1 and bile acid quantification were performed using the TargetLynx applications manager version 4.1 (Waters, Milford, Mass.).

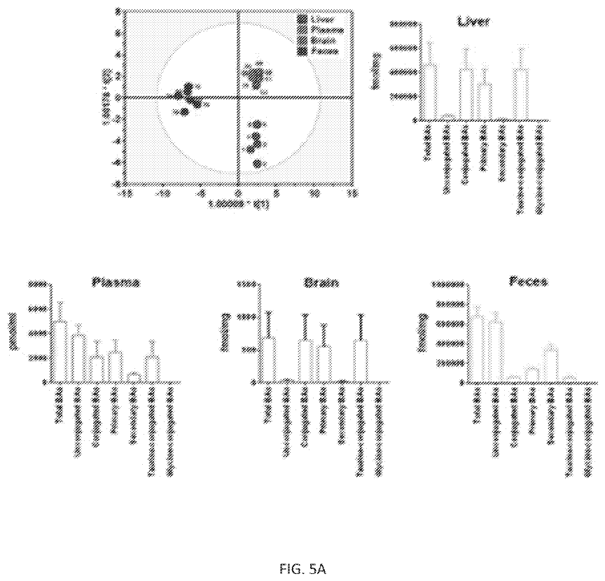

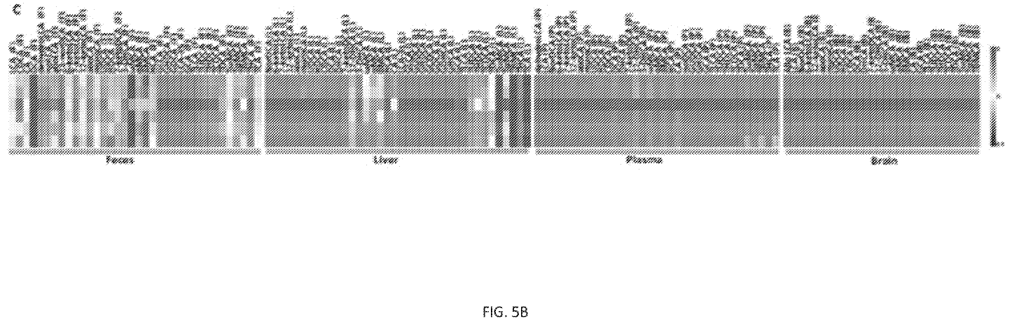

In plasma, 35 bile acids were detected, while 38 bile acids were detected in liver, 30 bile acids were detected in brain, and 36 bile acids were detected in feces. Principal component analysis (PCA) was performed to discriminate the bile acid profile among the four different compartments (liver, plasma, brain and feces). The resulting scores plots showed that the bile acid profiles in plasma and brain were similar but were different from the bile acid profiles in liver and feces (FIG. 5A). The amounts of total bile acids in feces were the highest, followed by liver, plasma and brain. The conjugated bile acids were rich in liver while the unconjugated bile acids were predominant in feces. The taurine-conjugated bile acids were predominant among the conjugated bile acids with much more amount than glycine-conjugated bile acids. The secondary bile acids were rich in feces while primary bile acids were rich in both liver and feces. The amount of bile acids in plasma and brain were much lower than in liver and feces (FIG. 5B). The heatmap also showed that the bile acid concentrations were significant different among the liver, plasma, brain and feces (FIG. 5C).

This mouse study provides a clear visualization of the bile acid profiles in liver, plasma, feces, and brain in normal mice. A total of 30 bile acids were detected in the mouse brain. These data suggest that the brain bile acids are derived from circulating bile acids in blood, as they are most significantly correlated with plasma profiles of bile acids, compared to the bile acid profiles in feces and liver (FIG. 5A). Brain bile acids are presumably transported across the blood-brain barrier from the blood.

### Example 6

### Bile Acid Profiles in Alzheimer's Disease

Binary logistic regression models were used to assess associations between bile acid levels and diagnostic groups at baseline. The Bonferroni-corrected threshold for statistical significance was determined as P<4.76×10⁻⁴(0.05 divided by 15 metabolites times 7 phenotypes including cognition). When bile acid profiles were compared in AD patients to those of CN subjects, a significant decrease in levels of the primary bile acid, CA (P=1.56×10⁻⁴) was detected. This observation was in contrast to a significant increase in levels of its product the secondary bacterially produced bile acid, DCA (P=1.61×10⁻⁴). The secondary conjugated bile acids, GDCA, TDCA, and GLCA, also showed significantly increased levels in AD (Table 6).

TABLE 6
Primary and Secondary bile acids measured in the ADNI study and their
cross-sectional association with diagnosis and cognition^{a}.

| | | | AD vs. CN (n = 673) | ADAS-Cog13 (n = 1453) |
|---|---|---|---|---|
| Abbreviation | Bile Acid Name | Category | OR (95% CI); P-value^{b} | β(95% CI); P-value^{c} |
| CA | Cholic | Primary | 0.85(0.78, 0.92); 1.56E−04 | −0.04(−0.07, −0.01); 2.81E−03 |
| CDCA | Chenodeoxycholic | Primary | 0.94(0.87, 1.01); 7.19E−02 | −0.02(−0.04, 0.00); 1.07E−01 |
| GCA | Glycocholic | Primary | 1.07(0.96, 1.18); 2.03E−01 | 0.01(−0.02, 0.05); 4.36E−01 |
| | | Conjugated | | |
| GCDCA | Glycochenodeoxycholic | Primary | 1.15(1.02, 1.29); 2.07E−02 | 0.06(0.02, 0.09); 4.60E−03 |
| | | Conjugated | | |
| TCA | Taurocholic | Primary | 1.03(0.94, 1.12); 5.44E−01 | −0.01(−0.03, 0.03); 7.84E−01 |
| | | Conjugated | | |
| TCDCA | Taurochenodeoxycholic | Primary | 1.04(0.94, 1.15); 4.29E−01 | 0.02(−0.02, 0.05); 3.39E−01 |
| | | Conjugated | | |
| TMCA | Tauromuricholic | Primary | 1.09(1.00, 1.18); 4.46E−02 | 0.029(0.00, 0.06); 4.21E−02 |
| | | Conjugated | | |
| DCA | Deoxycholic | Secondary | 1.24(1.11, 1.39); 1.61E−04 | 0.05(0.01, 0.08); 9.26E−03 |
| HDCA | Hyodeoxycholic | Secondary | Excluded | Excluded |
| LCA | Lithocholic | Secondary | Excluded | Excluded |
| UDCA | Ursodeoxycholic | Secondary | 0.96(0.90, 1.03); 2.41E−01 | −0.01(−0.03, 0.01); 2.44E−01 |
| GDCA | Glycodeoxycholic | Secondary | 1.30(1.17, 1.43); 4.20E−07 | 0.07(0.04, 0.10); 1.05E−05 |
| | | Conjugated | | |
| TDCA | Taurodeoxycholic | Secondary | 1.19(1.08, 1.30); 3.26E−04 | 0.05(0.02, 0.08); 2.39E−03 |
| | | Conjugated | | |
| GLCA | Glycolithocholic | Secondary | 1.33(1.20, 1.48); 9.21E−08 | 0.07(0.04, 0.11); 1.97E−05 |
| | | Conjugated | | |
| TLCA | Taurolithocholic | Secondary | 1.18(1.06, 1.30); 1.50E−03 | 0.06(0.03, 0.09); 4.83E−04 |
| | | Conjugated | | |
| GUDCA | Glycoursodeoxycholic | Secondary | 1.09(1.00, 1.19); 5.39E−02 | 0.03(−0.00, 0.06); 6.04E−02 |
| | | Conjugated | | |
| TUDCA | Tauroursodeoxycholic | Secondary | 1.08(0.96, 1.20); 1.86E−01 | 0.01(−0.02, 0.05); 4.85E−01 |
| | | Conjugated | | |

^{a}Statistically significant associations of metabolites with diagnosis or cognition are shaded in gray. The ones that surpassed the Bonferroni correction are bolded.
^{b}Odds ratios and p-values were obtained from logistics regressions. Models were corrected for age, sex, body mass index and APOE ε4 status; Bonferroni-adjusted critical value was set to 5.56E−04.
^{c}Outcome: Square root of ADASCog-13 (0 [best] to 85 [worst]); Models were corrected for age, sex, years of education, body mass index and APOE ε4 status; Bonferroni-adjusted critical value was set to 2.17E−03.

Similar differences and trends were also noted between AD cases and both MCI groups, while the comparison of bile acid levels between the CN and MCI groups yielded no significant results (Table 7).

TABLE 7
Levels and ratios of bile acids reflective of gut microbiome and liver enzymatic
activities and their correlation with disease status and cognitive function.

| | AD vs. CN | EMCI vs. AD | LMCI vs. AD |
|---|---|---|---|
| Metabolite | OR(95% CI); P | OR (95% CI); P | OR (95% CI); P |
| CA | 0.85(0.78-0.92); 1.56E−04 | 0.86(0.78-0.94); 1.75E−03 | 0.93(0.87-1.00); 6.55E−02 |
| CDCA | 0.94(0.87-1.01); 7.19E−02 | 0.94(0.87-1.01); 9.77E−02 | 0.97(0.91-1.03); 2.54E−01 |
| DCA | 1.24(1.11-1.39); 1.61E−04 | 1.14(1.02-1.27); 2.70E−02 | 1.16(1.05-1.28); 3.10E−03 |
| GCA | 1.07(0.96-1.18); 2.03E−01 | 1.05(0.94-1.18); 3.52E−01 | 1.06(0.97-1.16); 2.06E−01 |
| GCDCA | 1.15(1.02-1.29); 2.07E−02 | 1.11(0.98-1.25); 1.01E−01 | 1.10(0.99-1.22); 7.03E−02 |
| GDCA | 1.30(1.17-1.43); 4.20E−07 | 1.21(1.09-1.34); 2.56E−04 | 1.17(1.08-1.28); 2.53E−04 |
| GLCA | 1.33(1.20-1.48); 9.21E−08 | 1.16(1.04-1.29); 6.54E−03 | 1.21(1.11-1.33); 2.38E−05 |
| GUDCA | 1.09(1.00-1.19); 5.39E−02 | 1.07(0.97-1.18); 1.66E−01 | 1.04(0.97-1.13); 2.81E−01 |
| TCA | 1.03(0.94-1.12); 5.44E−01 | 1.01(0.92-1.11); 8.51E−01 | 1.03(0.96-1.12); 3.95E−01 |
| TCDCA | 1.04(0.94-1.15); 4.29E−01 | 1.03(0.93-1.14); 5.94E−01 | 1.02(0.94-1.12); 5.81E−01 |
| TDCA | 1.19(1.08-1.30); 3.26E−04 | 1.15(1.04-1.27); 5.21E−03 | 1.11(1.03-1.20); 8.55E−03 |
| TLCA | 1.18(1.06-1.30); 1.50E−03 | 1.21(1.09-1.35); 3.80E−04 | 1.10(1.01-1.20); 2.47E−02 |
| TMCA | 1.09(1.00-1.18); 4.46E−02 | 1.04(0.95-1.14); 3.75E−01 | 1.04(0.97-1.12); 2.80E−01 |
| TUDCA | 1.08(0.96-1.20); 1.86E−01 | 1.03(0.91-1.16); 6.62E−01 | 1.03(0.93-1.13); 6.08E−01 |
| UDCA | 0.96(0.90-1.03); 2.41E−01 | 0.89(0.83-0.95); 8.72E−04 | 1.00(0.95-1.05); 8.79E−01 |
| CA:CDCA | 0.87(0.77-0.97); 1.67E−02 | 0.92(0.82-1.04); 1.82E−01 | 0.97(0.88-1.06); 5.17E−01 |
| DCA:CA | 1.25(1.16-1.35); 1.53E−08 | 1.19(1.10-1.29); 3.93E−05 | 1.13(1.06-1.21); 2.86E−04 |
| GLCA:CDCA | 1.16(1.09-1.23); 3.61E−06 | 1.10(1.03-1.17); 2.82E−03 | 1.10(1.04-1.16); 5.08E−04 |
| GDCA:CA | 1.24(1.16-1.33); 8.53E−10 | 1.20(1.12-1.30); 1.01E−06 | 1.12(1.06-1.19); 9.74E−05 |
| GDCA:DCA | 1.16(1.02-1.31); 2.41E−02 | 1.17(1.03-1.34); 1.98E−02 | 1.09(0.97-1.22); 1.52E−01 |
| TDCA:CA | 1.16(1.10-1.24); 9.83E−07 | 1.15(1.07-1.23); 5.84E−05 | 1.08(1.03-1.14); 3.00E−03 |
| TLCA:CDCA | 1.09(1.03-1.16); 1.60E−03 | 1.10(1.04-1.17); 1.09E−03 | 1.05(1.00-1.10); 3.50E−02 |
| TDCA:DCA | 1.02(0.93-1.11); 7.40E−01 | 1.05(0.95-1.16); 3.65E−01 | 1.01(0.93-1.09); 8.59E−01 |

^{a}Statistically significant associations of metabolites with diagnosis or cognition are shaded in gray. The ones that surpassed the Bonferroni correction are bolded.
^{b}Odds ratios and p-values were obtained from logistics regressions. Models were corrected for age, sex, body mass index and APOE ε4 status; Bonferroni-adjusted critical value was set to 5.56E−04.
^{c}Outcome: Square root of ADASCog-13 (0 [best] to 85 [worst]); Models were corrected for age, sex, years of education, body mass index and APOE ε4 status; Bonferroni-adjusted critical value was set to 2.17E−03.

### Example 7

### Ratios Pertaining to Bile Acid Conversion by Gut Microbiota Linked to Alzheimer's Disease and Cognitive Decline

To determine which enzymatic processes in bile acid metabolism may underlie the changes noted in AD, eight selected ratios reflective of enzymatic activities in liver and in the gut microbiome we investigated. These ratios included: 1) CA:CDCA ratio to test for a possible shift in bile acid synthesis from the primary to the alternative bile acid pathway in the liver; 2) DCA:CA, GLCA:CDCA and TLCA:CDCA ratios to test for changes in gut microbiome enzymatic activity leading to altered production of secondary bile acids; 3) GDCA:DCA and TDCA:DCA ratios to test for enzymatic changes related to conjugation of secondary bile acids to its glycine and taurine forms.

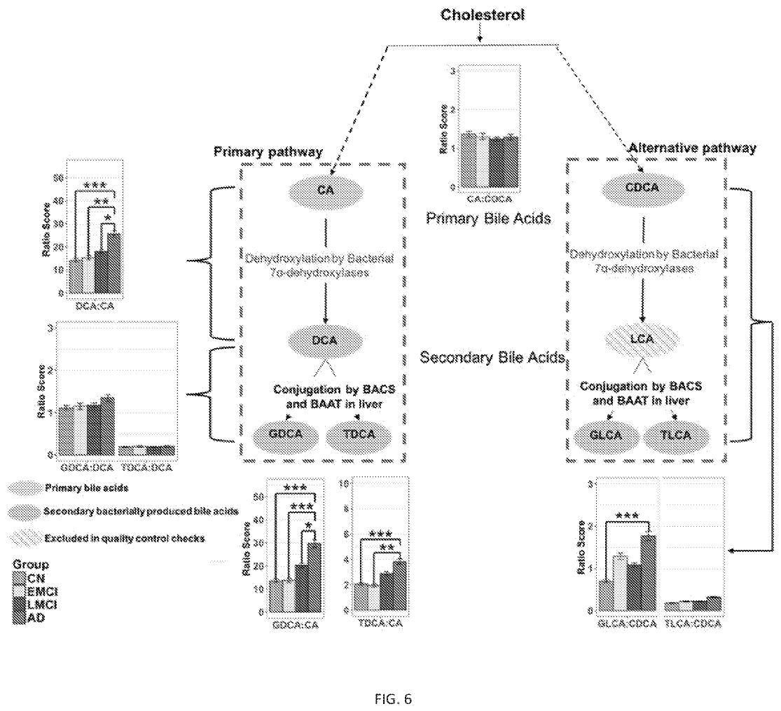

Associations were considered as significant at a Bonferroni-corrected P<3.11×10⁻⁴ (0.05 divided by all 23 metabolic traits times 7 phenotypes, which include cognitive function). The ratio of the primary bile acids (CA:CDCA) showed no significant association with AD. Yet, for the ratio of DCA:CA (the conversion of unconjugated primary to unconjugated secondary bile acid), a highly significant association with AD diagnosis was observed (P=1.53×10⁻⁸). Ratios between primary and secondary conjugated bile acids showed the same effect and directional trend, including GDCA:CA (P=8.53×10⁻¹⁰), TDCA:CA (P=9.83×1e) and GLCA:CDCA (P=3.61×10⁻⁶). Ratios modeling the glycine and taurine conjugation step of DCA (e.g., GDCA:DCA, TDCA:DCA) were not significantly linked to diagnosis (FIG. 6 and Table 8).

TABLE 8
Ratios of bile acids reflective of gut microbiome and liver enzymatic activities
and their correlation with disease status and cognitive function.

| Ratios informative about | Ratios | AD vs. CN (n = 673) | ADAS-Cog13 (n = 1453) |
|---|---|---|---|
| metabolic processes | calculated | OR (95% CI); P-value^{a} | p(95% CI); P-value^{b} |
| Bile acid synthesis. primary | CA:CDCA | 0.87(0.77, 0.97); 1.67E−02 | −0.03(−0.07, 0.01); 1.27E−01 |
| vs, alternative pathway | | | |
| Conversion from primary to | DCA:CA | 1.25(1.16, 1.35); 1.53E−08 | 0.05(0.03, 0.08); 1.05E−05 |
| secondary BA by the gut | GDCA:CA | 1.24(1.16, 1.33); 8.53E−10 | 0.06(0.04, 0.08); 1.20E−07 |
| microbiome | TDCA:CA | 1.16(1.10, 1.24); 9.83E−07 | 0.04(0.02, 0.06); 5.40E−05 |
| | LCA:CDCA | NA^{c} | NA^{c} |
| | GLCA:CDCA | 1.16(1.09, 1.23); 3.61E−06 | 0.04(0.02, 0.06); 9.15E−05 |
| | TLCA:CDCA | 1.09(1.03, 1.16); 1.60E−03 | 0.03(0.01, 0.05); 1.50E−03 |
| Glycine or Taurine | GDCA:DCA | 1.16(1.02, 1.31); 2.41E−02 | 0.05(0.02, 0.10); 5.49E−03 |
| conjugation of secondary | TDCA:DCA | 1.02(0.93, 1.11); 7.40E−01 | 0.01(−0.02, 0.04); 4.15E−01 |
| bile acids by liver enzymes | GLCA:LCA | NA^{c} | NA^{c} |
| | TLCA:LCA | NA^{c} | NA^{c} |

^{a}Several ratios were calculated to inform about possible enzymatic activity changes in Alzheimer's patients. These ratios reflect: (1) Shift in bile acid metabolism from primary to alternative pathway. (2) Changes in gut microbiome correlated with production of secondary bile acids. (3) Changes in glycine and taurine conjugation of secondary bile acids.
^{b}Outcome: Baseline diagnosis; Odds ratios and p-values were obtained from logistics regressions. Models were corrected for age, sex, body mass index and APOE ε4 status; Bonferroni-adjusted critical value was set to 1.04E−03 based on 6 possible pairwise comparison of diagnosis groups (CN, EMCI, LMCI and AD) for 8 ratios.
^{b}Outcome: Square root of ADASCog-13 (0 [best] to 85 [worst]); Models were corrected for age, sex, years of education, body mass index and APOE ε4 status; Bonferroni-adjusted critical value was set to 2.17E−03.
^{c}Ratio is not available as LCA was excluded in the preprocessing checks.

In order to extend this analysis to a quantitative phenotype making use of the full ADNI dataset, cross-sectional associations of bile profiles and ratios with the ADAS-Cog13 score as a measure of cognitive performance (higher values of ADAS-Cog13 indicate poorer cognition) were evaluated. Two secondary conjugated bile acids (GDCA, GLCA) and four ratios (including DCA:CA and GLCA:CDCA) were significantly associated with increases in ADAS-Cog13 scores (poorer cognition). For the ratios, the same pattern was observed as for AD diagnosis, with higher ratios of secondary to primary bile acids being highly significantly associated with cognitive function, while neither conjugation nor a shift between primary and alternative bile pathways in the liver could be significantly linked to cognition (Tables 5 and 7).

### Example 8

### Association of Blood and Brain Levels of DCA:CA with Cognitive Function in ROS/MAP Cohort

To verify the above results, experiments were replicated in the independent ROS/MAP study cohort. Because AD cases were underrepresented in the cohort, cognitive function was used as the dependent phenotype. In particular, experiments were conducted to determine if the association of the DCA:CA ratio was replicable (as proxy for bile acid processing by the gut microbiome) with respect to cognitive function, while adjusting for age, sex, copies of the APOE ε4 allele, and years of education. In both blood and brain samples, the association with the same directional tread as in ADNI was demonstrated (DCA:CA in blood:β=−0.06; P=0.011; DCA:CA in brain: β=−0.21; P=0.032).

### Example 9

### Genetic Risk Variants for Alzheimer's Disease Linking Immune Function with Bile Acid Levels

Genetic association studies in AD have robustly identified several genetic risk variants in immune-related genes. To investigate if risk variants in these genes may contribute to changes in gut microbiome composition resulting in the observed associations in bile acid metabolism, nine genetic AD risk variants in candidate genes with immune-related functions (ABI3, CLU, CR1, EPHA1, INPP5D, MEF2C, MS4A6A, PLCG2 and TREM2) were selected. These variants were tested for associations with levels of bile acids in the primary bile pathway (e.g., CA, DCA, GDCA, and TDCA) as well as the DCA:CA ratio in a subset of ADNI subjects and RS subjects. In addition, associations from a published cohort-based study were included to increase sample sizes. With exception of variant rs983392 in MS4A6A, significant associations were found for the candidate variants in all of these genes. Three associations were significant after Bonferroni-correction (P<1.1×10⁻³) in one of the studies: rs616338 (ABI3) and rs190982 (MEF2C) were significantly associated with the DCA:CA ratio, and rs11771145 (EPHA1) was significantly linked to both DCA and TDCA.

### Example 10

### Genetic Risk Variants for Alzheimer's Disease Linking Immune Function with Bile Acid Levels

To further investigate that increased levels of DCA caused by gut dysbiosis may trigger molecular downstream effects relevant to the pathogenesis of AD, potentially significant genetic associations with DCA levels (P<1.0×10) were collected in a large population-based cohort study. The resulting 13 loci were annotated with genetic trait associations, including AD associations from the IGAP study. Two genes, CYP7A1 and IMPA2, showed association with DCA levels in ADNI subjects. Another gene in the list, ABCA7, is an AD risk gene, and five additional genes (LRRC7, CYCS, GPC6, FOXN3 and CNTNAP4) have been previously linked via genetic studies to AD endophenotypes, including cognitive decline and CSF protein levels.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method for An assay for the detection or quantification of bile acids and bile acid derivatives in a biological sample from a subject, the assay comprising: quantifying levels of at least one bile acid and levels of a derivative of the at least one bile acid in a biological sample from a subject; and generating a bile acid profile based on the levels of the at least one bile acid and the levels of a derivative of the at least one bile acid in the biological sample.

Clause 1. The assay according to clause 1, wherein the bile acid profile is indicative of a neurological disorder selected from the group consisting of dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders.

Clause 3. The assay according to clause 1 or clause 2, wherein the neurological disorder is Alzheimer's Disease.

Clause 4. The assay according to any of clauses 1 to 3, wherein generating the bile acid profile comprises calculating a ratio of the at least one bile acid derivative to the at least one bile acid.

Clause 5. The assay according to any of clauses 1 to 4, further comprising determining that the subject has a neurological disorder when the ratio of the at least one bile acid derivative to the at least one bile acid exceeds 1.0.

Clause 6. The assay according to any of clauses 1 to 5, wherein the bile acid profile is indicative of at least one of a defect in composite memory function, a defect in executive functioning, an increase in Alzheimer's Disease Assessment Scale (ADAS-Cog 13) score, and an increase Spatial Pattern of Abnormality for Recognition of Early Alzheimer's disease (SPARE-AD).

Clause 7. The assay according to any of clauses 1 to 6, wherein the bile acid profile is indicative of an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio.

Clause 8. The assay according to any of clauses 1 to 7, wherein the bile acid profile is indicative of at least one of an increase in brain ventricular volume, an increase in brain atrophy, a decrease in brain cortical thickness, and a decrease in brain glucose metabolism.

Clause 9. The assay according to any of clauses 1 to 7, wherein the at least one bile acid is cholic acid (CA) and the at least one bile acid derivative is deoxycholic acid (DCA).

Clause 10. The assay according to any of clauses 1 to 7, wherein the at least one bile acid is deoxycholic acid (DCA) and the at least one bile acid derivative is glucodeoxycholic acid (GDCA).

Clause 11. The assay according to any of clauses 1 to 7, wherein the at least one bile acid is chenodeoxycholic acid (CDCA) and the at least one bile acid derivative is glycochenodeoxycholic acid (GCDCA).

Clause 12. The assay according to any of clauses 1 to 7, wherein the at least one bile acid is chenodeoxycholic acid (CDCA) and the at least one bile acid derivative is taurochenodeoxycholic acid (TCDCA).

Clause 13. The assay according to any of clauses 1 to 7, wherein the at least one bile acid is chenodeoxycholic acid (CDCA) and the at least one bile acid derivative is glycolithocholic acid (GLCA).

Clause 14. The assay according to any of clauses 1 to 7, wherein the at least one bile acid is chenodeoxycholic acid (CDCA) and the at least one bile acid derivative is taurolithocholic acid (TLCA).

Clause 15. The assay according to any of clauses 1 to 7, wherein the at least one bile acid is ursodeoxycholic acid (UDCA) and the at least one bile acid derivative is glycoursodeoxycholine acid (GUDCA).

Clause 16. The assay according to any of clauses 1 to 15, wherein the biological sample from the subject is at least one of whole blood, serum, plasma, and cerebral spinal fluid (CSF).

Clause 17. The assay according to any of clauses 1 to 16, wherein quantifying levels of at least one primary bile acid and levels of at least one primary bile acid derivative comprises at least one of liquid chromatography (LC), ultra-high pressure liquid chromatography (UPLC), tandem mass spectrometry (MS), liquid chromatography tandem mass spectrometry (LC-MS-MS), and triple quadrupole tandem mass spectrometer operated in Multiple Reaction Monitoring (MRM) mode.

Clause 18. A method for treating a neurological disorder in a subject, the method comprising: administering a composition comprising a bile acid modulating agent to the subject, wherein the bile acid modulating agent modulates the function of at least one of Farnesoid X Receptor (FXR) and G Protein Coupled Bile Acid Receptor 1 (GPBAR1/TGR5).

Clause 19. The method according to clause 18, wherein the administration of the composition treats the neurological disorder by ameliorating at least one symptom of the neurological disorder, wherein the at least one symptom of the neurological disorder is selected from the group consisting of: a defect in composite memory function; a defect in executive functioning; an increase in Alzheimer's Disease Assessment Scale (ADAS-Cog 13) score; an increase Spatial Pattern of Abnormality for Recognition of Early Alzheimer's disease (SPARE-AD); an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio; an increase in brain ventricular volume; an increase in brain atrophy; a decrease in brain cortical thickness; and a decrease in brain glucose metabolism.

Clause 20. The method according to clause 18 or clause 19, further comprising: quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample prior to the administration of the composition.

Clause 21. The method according to any of clauses 18 to 20, wherein the bile acid modulating agent is selected from the group consisting of: i) FXR agonists selected from the group consisting of Obeticholic acid, OCA, 1NT-747, 1NT-767, GW4064, GSK2324, PX-102, PX20606, GS9674, Way362362450, and fexaramine and LJN452; ii) TGR5 agonists selected from the group consisting of 1NT-767, BAR502, and 1NT-777; iii) FGF-19 analogue, NGM-282; iv) ASBT inhibitors selected from the group consisting of LUM-001, A4250, and GSK2330672; v) PPAR agonists selected from the group consisting of fenofibrate, bezafibrate and GFT505; vi) UDCA-related compounds selected from the group consisting of norUDCA and Tauroursodeoxycholate (TUDCA); vii) Fatty acid-bile acid conjugate, Aramchol; viii) Resins selected from the group consisting of colestipol, colesevelam, colestimide, and sevelamer; ix) NTCP inhibitor, Myrcludex B; and x) any combinations thereof.

Clause 22. A method of aiding in the determination of whether a subject has a neurological disorder, the method comprising: quantifying levels of at least one bile acid and levels of a derivative of the at least one bile acid in a biological sample from a subject; calculating the ratio of the at least one bile acid derivative to the at least one bile acid; and determining that the subject has a neurological disorder when the ratio of the at least one bile acid derivative to the at least one bile acid exceeds 1.0.

Clause 23. The method according to clause 22, wherein the at least one bile acid is cholic acid (CA) and the at least one bile acid derivative is deoxycholic acid (DCA).

Clause 24. The method according to clause 22, wherein the at least one bile acid is deoxycholic acid (DCA) and the at least one bile acid derivative is glucodeoxycholic acid (GDCA).

Clause 25. The method according to clause 22, wherein the at least one bile acid is chenodeoxycholic acid (CDCA) and the at least one bile acid derivative is glycochenodeoxycholic acid (GCDCA).

Clause 26. The method according to clause 22, wherein the at least one bile acid is chenodeoxycholic acid (CDCA) and the at least one bile acid derivative is taurochenodeoxycholic acid (TCDCA).

Clause 27. The method according to clause 22, wherein the at least one bile acid is chenodeoxycholic acid (CDCA) and the at least one bile acid derivative is glycolithocholic acid (GLCA).

Clause 28. The method according to clause 22, wherein the at least one bile acid is chenodeoxycholic acid (CDCA) and the at least one bile acid derivative is taurolithocholic acid (TLCA).

Clause 29. The method according to clause 22, wherein the at least one bile acid is ursodeoxycholic acid (UDCA) and the at least one bile acid derivative is glycoursodeoxycholine acid (GUDCA).

Clause 30. The method according to any of clauses 22 to 29, wherein the neurological disorder comprises at least one of dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders.

Clause 31. The method according to any of clauses 22 to 29, wherein the neurological disorder is Alzheimer's Disease.

Clause 32. The method according to any of clauses 22 to 31, wherein the biological sample from the subject is at least one of whole blood, serum, plasma, and cerebral spinal fluid (CSF).

Clause 33. The method according to any of clauses 22 to 32, wherein quantifying levels of at least one primary bile acid and levels of at least one primary bile acid derivative comprises at least one of liquid chromatography (LC), ultra-high pressure liquid chromatography (UPLC), tandem mass spectrometry (MS), liquid chromatography tandem mass spectrometry (LC-MS-MS), and triple quadrupole tandem mass spectrometer operated in Multiple Reaction Monitoring (MRM) mode.

Clause 34. The method according to any of clauses 22 to 33, wherein the method further comprises performing a neurological assessment of the subject to verify presence of at least one independent indicator of the neurological disorder.

Clause 35. The method according to any of clauses 22 to 34, wherein the neurological assessment comprises at least one of a neuroimaging procedure, determining a Alzheimer's Disease Assessment Scale cognitive subscale 13 (ADAS-Cog 13) score, determining a Spatial Pattern of Abnormality for Recognition of Early Alzheimer's disease (SPARE-AD) score, measurement of executive function, measurement of memory function, measurement of brain ventricular volume, measurement of brain atrophy, measurement of cortical thickness, measurement of Amyloid β1-42 protein fragment (Aβ₁₋₄₂), measurement of total Tau (T-tau)/Aβ₁₋₄₂ ratio, and combinations thereof.

Clause 36. The method according to clause 35, wherein the at least one independent neurological indicator correlates with levels of the at least one primary bile acid or levels of the at least one primary bile acid derivative indicating the presence of the neurological disorder.

Clause 37. A biomarker panel for aiding in the determination of whether a subject has a neurological disorder, the panel comprising: at least one primary bile acid biomarker and at least one primary bile acid derivative biomarker; wherein quantifying levels of the at least one primary bile acid biomarker and the at least one primary bile acid derivative biomarker aids in the determination of whether the subject has a neurological disorder.

Clause 38. The panel according to clause 37, wherein the at least one primary bile acid biomarker is selected from the group consisting of cholic acid (CA) and chenodeoxycholic acid (CDCA); and wherein the at least one primary bile acid derivative biomarker is selected from the group consisting of deoxycholic acid (DCA), glycodeoxycholic acid (GDCA), and glycochenodeoxycholic acid (GCDCA).

Clause 39. The panel according to clause 37 or clause 38, further comprising at least one of the following biomarkers or a variant thereof: ABI3, CLU, CR1, EPHA1, INPP5D, MEF2C, MS4A6A, PLCG2, TREM2, CYP7A1, IMPA2, LRRC7, CYCS, GPC6, FOXN3, and CNTNAP4.

1. An assay for the detection or quantification of bile acids and bile acid derivatives in a biological sample from a subject, the assay comprising:
quantifying levels of at least one bile acid and levels of a derivative of the at least one bile acid in a biological sample from a subject; and
generating a bile acid profile based on the levels of the at least one bile acid and the levels of a derivative of the at least one bile acid in the biological sample.

2. The assay according to claim 1, wherein the bile acid profile is indicative of a neurological disorder selected from the group consisting of dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders.

3. (canceled)

4. The assay according to claim 1, wherein generating the bile acid profile comprises calculating a ratio of the at least one bile acid derivative to the at least one bile acid.

5. The assay according to claim 4, further comprising determining that the subject has a neurological disorder when the ratio of the at least one bile acid derivative to the at least one bile acid exceeds 1.0.

6. The assay according to claim 1, wherein the bile acid profile is indicative of at least one of a defect in composite memory function, a defect in executive functioning, an increase in Alzheimer's Disease Assessment Scale (ADAS-Cog 13) score, and an increase Spatial Pattern of Abnormality for Recognition of Early Alzheimer's disease (SPARE-AD).

7. The assay according to claim 1, wherein the bile acid profile is indicative of an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio.

8. The assay according to claim 1, wherein the bile acid profile is indicative of at least one of an increase in brain ventricular volume, an increase in brain atrophy, a decrease in brain cortical thickness, and a decrease in brain glucose metabolism.

9. The assay according to claim 1, wherein the at least one bile acid is cholic acid (CA) and the at least one bile acid derivative is deoxycholic acid (DCA),
wherein the at least one bile acid is deoxycholic acid (DCA) and the at least one bile acid derivative is glucodeoxycholic acid (GDCA),
wherein the at least one bile acid is chenodeoxycholic acid (CDCA) and the at least one bile acid derivative is glycochenodeoxycholic acid (GCDCA),
wherein the at least one bile acid is chenodeoxycholic acid (CDCA) and the at least one bile acid derivative is taurochenodeoxycholic acid (TCDCA),
wherein the at least one bile acid is chenodeoxycholic acid (CDCA) and the at least one bile acid derivative is glycolithocholic acid (GLCA),
wherein the at least one bile acid is chenodeoxycholic acid (CDCA) and the at least one bile acid derivative is taurolithocholic acid (TLCA), or
wherein the at least one bile acid is ursodeoxycholic acid (UDCA) and the at least one bile acid derivative is glycoursodeoxycholine acid (GUDCA).

10-17. (canceled)

18. A method for treating a neurological disorder in a subject, the method comprising:
administering a composition comprising a bile acid modulating agent to the subject, wherein the bile acid modulating agent modulates the function of at least one of Farnesoid X Receptor (FXR) and G Protein Coupled Bile Acid Receptor 1 (GPBAR1/TGR5).

19. The method according to claim 18, wherein the administration of the composition treats the neurological disorder by ameliorating at least one symptom of the neurological disorder, wherein the at least one symptom of the neurological disorder is selected from the group consisting of:
a defect in composite memory function; a defect in executive functioning; an increase in Alzheimer's Disease Assessment Scale (ADAS-Cog 13) score; an increase Spatial Pattern of Abnormality for Recognition of Early Alzheimer's disease (SPARE-AD); an increase in at least one of Amyloid β1-42 (Aβ₁₋₄₂) levels, total Tau levels, phosphorylated Tau levels, fibrillary Tau levels, and Tau (T-tau)/Aβ₁₋₄₂ ratio; an increase in brain ventricular volume; an increase in brain atrophy; a decrease in brain cortical thickness; and a decrease in brain glucose metabolism.

20. The method according to claim 18, further comprising:
quantifying levels of at least one bile acid and levels of a derivative of the at least one bile acid in a biological sample from a subject, and generating a bile acid profile based on the levels of the at least one bile acid and the levels of a derivative of the at least one bile acid in the biological sample prior to the administration of the composition.

21. The method according to claim 18, wherein the bile acid modulating agent is selected from the group consisting of:
i) FXR agonists selected from the group consisting of Obeticholic acid, OCA, INT-747, INT-767, GW4064, GSK2324, PX-102, PX20606, GS9674, Way362362450, and fexaramine and LJN452;
ii) TGR5 agonists selected from the group consisting of INT-767, BAR502, and INT-777;
iii) FGF-19 analogue, NGM-282;
iv) ASBT inhibitors selected from the group consisting of LUM-001, A4250, and GSK2330672;
v) PPAR agonists selected from the group consisting of fenofibrate, bezafibrate and GFT505;
vi) UDCA-related compounds selected from the group consisting of norUDCA and Tauroursodeoxycholate (TUDCA);
vii) Fatty acid-bile acid conjugate, Aramchol;
viii) Resins selected from the group consisting of colestipol, colesevelam, colestimide, and sevelamer;
ix) NTCP inhibitor, Myrcludex B; and
x) any combinations thereof.

22. A method of aiding in the determination of whether a subject has a neurological disorder, the method comprising:
quantifying levels of at least one bile acid and levels of a derivative of the at least one bile acid in a biological sample from a subject;
calculating the ratio of the at least one bile acid derivative to the at least one bile acid; and
determining that the subject has a neurological disorder when the ratio of the at least one bile acid derivative to the at least one bile acid exceeds 1.0.

23. The method according to claim 22, wherein the at least one bile acid is cholic acid (CA) and the at least one bile acid derivative is deoxycholic acid (DCA),
wherein the at least one bile acid is deoxycholic acid (DCA) and the at least one bile acid derivative is glucodeoxycholic acid (GDCA),
wherein the at least one bile acid is chenodeoxycholic acid (CDCA) and the at least one bile acid derivative is glycochenodeoxycholic acid (GCDCA),
wherein the at least one bile acid is chenodeoxycholic acid (CDCA) and the at least one bile acid derivative is taurochenodeoxycholic acid (TCDCA),
wherein the at least one bile acid is chenodeoxycholic acid (CDCA) and the at least one bile acid derivative is glycolithocholic acid (GLCA),
wherein the at least one bile acid is chenodeoxycholic acid (CDCA) and the at least one bile acid derivative is taurolithocholic acid (TLCA), or
wherein the at least one bile acid is ursodeoxycholic acid (UDCA) and the at least one bile acid derivative is qlycoursodeoxycholine acid (GUDCA).

24-29. (canceled)

30. The method according to claim 22, wherein the neurological disorder comprises at least one of dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, and HIV-associated neurocognitive disorders.

31-33. (canceled)

34. The method according to claim 22, wherein the method further comprises performing a neurological assessment of the subject to verify presence of at least one independent indicator of the neurological disorder.

35. (canceled)

36. The method according to claim 34, wherein the at least one independent neurological indicator correlates with levels of the at least one primary bile acid or levels of the at least one primary bile acid derivative indicating the presence of the neurological disorder.

37. A biomarker panel for aiding in the determination of whether a subject has a neurological disorder, the panel comprising:
at least one primary bile acid biomarker and at least one primary bile acid derivative biomarker;
wherein quantifying levels of the at least one primary bile acid biomarker and the at least one primary bile acid derivative biomarker aids in the determination of whether the subject has a neurological disorder.

38. The panel according to claim 37, wherein the at least one primary bile acid biomarker is selected from the group consisting of cholic acid (CA) and chenodeoxycholic acid (CDCA); and wherein the at least one primary bile acid derivative biomarker is selected from the group consisting of deoxycholic acid (DCA), glycodeoxycholic acid (GDCA), and glycochenodeoxycholic acid (GCDCA).

39. The panel according to claim 38, further comprising at least one of the following biomarkers or a variant thereof: ABI3, CLU, CR1, EPHA1, INPP5D, MEF2C, MS4A6A, PLCG2, TREM2, CYP7A1, IMPA2, LRRC7, CYCS, GPC6, FOXN3, and CNTNAP4.
